SEQUENCE LISTING (4 sequences)

SEQ ID NO: 1 - DNA, 20 bp, Homo sapiens
tggaggtgagacccaaagaa

SEQ ID NO: 2 - DNA, 21 bp, Homo sapiens
ctcagctacctgtgatgccga

SEQ ID NO: 3 - DNA, 23 bp, Homo sapiens
agactcacctctgttcagggaaa

SEQ ID NO: 4 - DNA, 16 bp, Homo sapiens
caccttggaagttggc

(19) United States
(12) Patent Application Publication
(10) Pub. No.: US 2002/0168623 A1
(43) Pub. Date: Nov. 14, 2002

(54) Compositions and methods for induction of proteins involved in xenobiotic metabolism

(76) Inventor: Judy Raucy, San Diego, CA (US)

Correspondence Address: DAVID R PRESTON & ASSOCIATES, 12625 HIGH BLUFF DRIVE, SUITE 205, SAN DIEGO, CA, 92130, US

(21) Appl. No.: 09/832,621

(22) Filed: Apr. 11, 2001

Related U.S. Application Data
(60) Provisional application No. 60/196,681, filed on Apr. 12, 2000.
(60) Provisional application No. 60/241,391, filed on Oct. 17, 2000.

(51) Int. Cl. C12Q 1/00; C12N 5/08; C12N 5/06

(52) U.S. Cl. 435/4; 435/325; 435/366

(57) ABSTRACT

The present invention provides improved cells and methods for identifying compounds that alter protein expression, such as xenobiotics, endobiotics, chemicals or drugs. The invention provides other benefits as well. One aspect of the present invention is a cell that includes a first nucleic acid molecule that includes: a promoter or enhancer operable for a nucleic acid molecule encoding a protein involved in drug metabolism (such as an enzyme or transporter) and a reporter gene; and a second nucleic acid encoding an intracellular receptor or transcription factor; so that when the intracellular receptor or transcription factor is bound with a compound, the intracellular receptor, transporter or transcription factor can operably bind with the promoter or enhancer resulting in the expression of said reporter gene. Another aspect of the present invention is a method for evaluating compounds for the property of inducing the expression of a gene encoding a protein involved in drug metabolism, including; providing a test compound; contacting the test compound with a cell of the present invention; and detecting the expression of said reporter gene. This method can be a high throughput method.

The present application claims benefit of priority to United States provisional patent application serial No. 60/196,681 entitled “Method for In Vitro Screening for Drug Metabolism” filed on Apr. 12, 2000, and United States provisional patent application serial No. 60/241,391 entitled “High Volume Screening for p450 Induction” filed Oct. 17, 2000, each of which is incorporated by reference herein in their entirety.

This invention was made with government support awarded by the National Institutes of Health, grant number GM-58287 The United States Government may have certain rights in the invention.

### TECHNICAL FIELD

The invention relates to the field of identifying compounds that alter expression of proteins.

### BACKGROUND

Adverse reactions to therapeutic agents are a common cause of morbidity and mortality, particularly in industrialized nations where the use of such therapeutic agents is relatively common. It has been estimated that side affects from drugs are the fourth to sixth leading cause of death in hospitals in the United States (Moore and Kliewer, Toxicology 153:1-10 (2000)). A large number of these adverse reactions are due to drug interactions, a process by which the administration of one drug alters the properties of a second co-administered drug. The most common drug interactions occur when one drug either increases or decreases the effectiveness of another (Moore and Kliewer, Toxicology 153:1-10 (2000)). This modification in the pharmacological action of a drug generally stems from alternations in the drug's metabolism. Thus, a major factor associated with drug interactions is altered metabolism.

The tissues most relevant to drug metabolism are the liver and intestine. Within these tissues the oxidative metabolism of drugs and other xenobiotics occurs through the action of a super-family of heme containing monooxygenases, collectively known as cytochrome P450 enzymes (CYP's) (Nebert and Gonzalez, Ann. Rev. Biochem. 56:945-993 (1987)). In general, the enzymatic actions of CYPs results in the formation of products with greater polarity, causing more rapid elimination of the product relative to the drug itself. This process can significantly alter a drug's pharmacodynamic profile. Such reactions are particularly important when they affect drugs with narrow therapeutic ranges.

The most abundant CYP enzyme present in the human liver and intestine is CYP3A4, accounting for about 70% of total enterocyte CYPs (Moore and Kliewer, Toxicology 153:1-10 (2000)) and about 29% to about 60% of hepatic P450s (Wrighton et al., Drug Metab. Rev. 32:339-361 (2000)). Substrates for CYP3A4, a microsomal enzyme, are generally highly lipophilic. The structural divergence of known CYP3A4 substrates is wide and includes endogenous steroids, contraceptive steroids, immunosuppressive agents, imidazole antimycotics and macrolide antibiotics (Wrighton et al., Drug Metab. Rev. 32:339-361 (2000)). Because of the abundance of CYP3A4 in liver and intestine and its broad substrate specificity, CYP3A4 is believed to play a dominant role in drug biotransformation. It is estimated that this P450 enzyme is involved in the metabolism of greater than 50% of all drugs in use today (Wrighton et al., Drug Metab. Rev. 32:339-361 (2000)).

Prolonged exposure to drugs can lead to an increased expression of specific p450s that can augment the metabolism and clearance of therapeutic drugs. CYP3A4 activity is enhanced by a range of diverse chemicals and its induced expression is the cause of many drug interactions.

Several of the most efficacious inducers of CYP3A4 expression are commonly used drugs such as the gluccocorticoid dexamethasone, the anticonvulsant phenobarbital, the antibiotic rifampicin and the antimycotic clotrimazone (Lehmann et al., J. Clin. Invest. 102:1016-1023 (1998)). As a result of elevated CYP3A4 levels, therapeutics metabolized by this P450 exhibit lower efficacy.

Therefore, it is important to identify agents possessing the ability to induce drug-metabolizing enzymes including, but not limited to, CYP3A4 and other p450 enzymes.

The biochemistry of P450 regulation can be complex. Some inducers of p450 activity have been identified. The levels of CYP3A4 are induced by exposure to a number of structurally diverse agents. This diversity can make it difficult to predict new drugs that may affect expression of that enzyme. Glucocorticoids and other nonsteroidal inducers of CYP3A4 may transcriptionally regulate the expression of this P450 by a mechanism involving an orphan nuclear receptor, pregnane X receptor (PXR) and potentially other receptors. PXR was identified as a new member of the nuclear hormone receptor super family. PXR mediates high dose glucocorticoid and pregnane steroid induction of the CYP3A4 promoter by heterodimerizing with the nuclear hormone receptor partner RXR and binding to an element highly conserved in the CYP3A4 promoter, the PXR element (PXRE). The nucleotide constraints for PXR binding have apparently been defined as AGTTCA arranged as a direct repeat (DR) or everted repeat (ER) with three, four, five or six nucleotide spacing (Wrighton et al., Drug Metab. Rev. 32:339-361 (2000)).

Because CYP3A4 and other CYPs can exhibit species differences, pharmaceutical companies test their drug candidates in vitro in human systems in order to gain an assessment of the potential for drug interactions in humans. Most in vitro testing involves the use of primary cultures of human hepatocytes. The availability of hepatocytes has afforded the pharmaceutical industry the ability to obtain clinically relevant in vitro drug interaction data. Compounds that are identified as potential inducers of a human P450 in hepatocytes can be screened out of further development, helping to alleviate the potential for a drug interaction and hence a safety and marketing liability (Rodrigues, Pharm. Res. 14:1504-1510 (1997)).

There are, however, disadvantages to utilizing primary cultures for these tests. One logistical problem with hepatocyte preparations is that enzymatic activities are not stable for longer than about four or five days. Also, these systems are costly, time consuming and produce variable responses. Furthermore, availability can be sporadic because primary cultures rely on the availability of human organs. Results obtained using these cells are also dependent upon culture media and conditions of culture. Finally, a limited number of compounds can be tested at any given time. This is particularly problematic because large numbers of candidate drugs are being produced through combinatorial chemistry and combinatorial biology methods.

Because of the inherent problems associated with the use of human hepatocytes for preclinical drug development, and the difficultly in obtaining liver specimens for research purposes, other in vitro systems are being investigated. Transcriptional activation has been performed in vitro for a number of years to investigate changes in gene expression of P450 enzymes by chemicals (Plant et al., Analyt. Biochem. 278:170-174 (2000)). The most common type is to use transient transfections of a reporter gene construct into a suitable cell line. This is then followed by dosing with a test compound, measuring of reporter gene production and comparison to control cells. At each step of this protocol, biological and experimental variations may be present which can provide poorly reproducible results and potentially erroneous interpretations. Examples of such variations include initial transfection efficiency, activation by factors endogenous to the host cell line and chemical specific effects such as cytotoxicity or proliferative effects. These problems decrease enthusiasm for using these types of systems.

### BRIEF DESCRIPTION OF THE FIGURES

FIG. 1 depicts a series of figures for one aspect of the present invention, where the first nucleic acid molecule and second nucleic acid molecule are provided as extra chromosomal elements such as plasmids. As depicted in FIG. 1A, a regulatory element P2 modulates the transcription of the gene encoding an intracellular receptor or transcription factor. The translation product can then interact with a test compound that binds with the intracellular receptor or transcription factor. As depicted in FIG. 1B, the complex of the intracellular receptor or transcription factor and xenobiotic or test compound can then bind with the promoter or enhancer operable for a nucleic acid molecule encoding a protein involved in drug metabolism. The complex can also enter the nucleus and optionally bind with the endogenous promoter or enhancer operable for a nucleic acid molecule encoding a protein involved in drug metabolism, if present or active in such cell. Upon binding of this complex with the promoter or enhancer operable for a nucleic acid molecule encoding a protein involved in drug metabolism, a reporter gene is transcribed and translated into a reporter and optionally the endogenous enzyme involved in drug metabolism is expressed, if present or active in such cell (FIG. 1C). That reporter can be detectable by its physical properties, such as fluorescence or luminescence, or can be a protein that is detectable based on its enzymatic conversion of substrate to product, such as a detectable product (FIG. 1D). Such reporters can be intracellular or extracellular. In another aspect of the present invention, both the first nucleic acid molecule and the second nucleic acid molecule are provided on the same extra chromosomal element, such as a single plasmid or YAC.

FIG. 2 depicts the case where the first nucleic acid molecule is an extra chromosomal element (20) whereas the second nucleic acid molecule is endogenous to the chromosome of the cell (22).

FIG. 3 depicts the case where the first nucleic acid molecule is an extra chromosomal element (30) and the second nucleic acid molecule is an exogenous nucleic acid molecule integrated into the genome of the cell (32).

FIG. 4 depicts the case where the first nucleic acid molecule is an exogenous nucleic acid molecule integrated into the genome of the cell (40) and the second nucleic acid molecule is endogenous to the chromosome of the cell (42).

FIG. 5 depicts the case where the first nucleic acid molecule is an exogenous nucleic acid molecule integrated into the genome of the cell (50) and the second nucleic acid molecule is an exogenous nucleic acid molecule integrated into the genome of the cell (52).

FIG. 6 depicts the construction of a HepG2 cell line containing the stably integrated CYP3A4 PXRE/luciferase reporter construct. The CYP3A4 PXRE containing the ER6 DNA sequence is provided in plasmid pGL3 Promoter (Promega). The transfected cells are cultured in the presence of geneticin to select for the integrated plasmid pIRESneo (Clonetech). Geneticin resistant colonys are screened for rifampicin enhanced light emission.

FIG. 7 depicts the response of the HepG2 cell line depicted in FIG. 6. Upon binding to a ligand such as a new chemical entity (NCE), endogenous HepG2 PXR is activated and forms a heterodimer with endogenous HepG2 RXR. The PXR/RXR complex binds to the PXRE sequence that was cloned into pGL3-Promoter (Promega) and stably integrated into the HepG² genome. Binding of the PXR/RXR complex activates transcription from the SV40 promoter of the integrated pGL3 Promoter plasmid. The luciferase gene is transcribed and translated causing the NCE dose-dependent emission of light.

FIG. 8 depicts a Northern blot analysis of RNA isolated from individual colonies stably transformed with pIRES containing hPXR alone (colonies D6, D4, B6, B5, A5, A2, W6, W5, W4, W2 and W1) or in combination of pGL3 promoter containing the CYP3A4 enhancer element described in the Examples (colony 6G). Each well contains 10 micrograms of total RNA and was developed with a cDNA probe specific for HPXR.

FIG. 9 depicts the effect of rifampicin and DMSO treatment on cells stably transformed with pGL3-promoter/3A4 enhancer. A 96 well plate was used to determine the length of exposure to produce high levels of induction of luciferase. Cells were treated between zero and seventy-eight hours prior to measuring luciferase activity. Results are expressed as fold increase over DMSO control cells and are the results of quadruplicate experiments.

FIG. 10 depicts the effect of rifampicin treatment on cells containing pGL3/3A4 enhancer plus phPXR. Cells were plated in a 96 well plate format and exposed to 10 micro molar rifampicin or DMSO for seventy-two hours. Results are expressed as relative light units and are the result of quadruplicate experiments.

FIG. 11 depicts various amounts of cells containing either hPXR plus pGL3/3A4 enhancer or the pGL3/3A4 enhancer alone were added to a 96 well plate and treated with 10 micro molar rifampicin or DMSO for forty-eight hours. Results are expressed as fold increase above control DMSO treated cells and are the result of quadruplicate experiments.

FIG. 12 depicts the effect of serum, DMSO and rifampicin on luciferase activity in HepG2 cells stably transformed with the pGL3 vector and pIRES vector with hPXR. Cells were treated for various time periods ranging from zero to seventy-eight hours in the presence or absence of rifampicin, DMSO or 0.1% serum in the media. An additional control without either rifampicin or DMSO was also included. Results are expressed as relative light units and are the result of quadruplicate experiments.

FIG. 13 depicts the effect of various CYP3A4 inducers on CYP3A4 expression in human hepatocytes. Human hepatocytes were exposed to 10 micro molar dexamethasone, no dexamethasone or the amount of dexamethasone normally present in hepatocyte culture media (about 10⁻⁷ M). Other inducers include one milli molar phenobarbital, ten micro molar rifampicin, clotrimazole or RU486. Total RNA (ten micrograms) was subjected to northern blot analysis and developed with a specific cDNA probe to CYP3A4 as described in the Examples.

FIG. 14 depicts the effects of various CYP3A4 inducers and non-inducers on HepG2 cells stably transformed with hPXR in pIRES vector and the 3A4 enhancer in the luciferase vector (colony IF). Cells were exposed to each inducer for seventy-two hours in a 96 well plate format prior to determining luciferase activity. Cells were treated with one micro molar dexamethasone, one hundred micro molar omeprazole, ten micro molar clotrimazole, ten micro molar RU486, ten micro molar rifampicin, one hundred micro molar mevastatin, fifty micro molar PCN, one hundred micro molar phenobarbital, one micro molar TCDD. Data is expressed as fold increase in luciferase activity above that in control DMSO treated cells and represents quadruplicate determinations.

FIG. 15 depicts the effects of various CYP3A4 inducers and non-inducers on HepG2 cells stably transformed with the 3A4 enhancer in the luciferase vector (colony 13). Cells were exposed to each inducer for seventy two hours in a 96 well plate format prior to determining luciferase activity. Cells were treated with one micro molar dexamethasone, one hundred micro molar omeprazole, ten micro molar clotrimazole, ten micro molar RU486, ten micro molar rifampicin, one hundred micro molar mevastatin, fifty micro molar PCN, one hundred micro molar phenobarbital, one micro molar TCDD. Data is expressed as fold increase in luciferase activity above that in control DMSO treated cells and represents quadruplicate determinations.

FIG. 16 depicts the effects of various doses of different CYP inducers on HepG2 cells stably transformed with the CYP3A4-enhancer in the luciferase vector (colony 13). Cells were exposed to each inducer for seventy two hours in a 96-well plate format prior to determining luciferase activity. Cells were treated with three doses of each drug. Doses ranged from 0.1 micromolar to 5 millimolar, depending on the agent. For dexamethasone doses were 0.1 micromolar, 1.0 micromolar and 10 micromolar; for omeprazole 50 micromolar, 100 micromolar and 250 micromolar; for clotrimazole 5 micromolar, 10 micromolar and 50 micromolar; for phenobarbital 1 millimolar, 2 millimolar and 5 millimolar; for TCDD 0.5 nanomolar, 1 nanomolar and 2 nanomolar; for RU486 5 micromolar, 10 micromolar and 50 micromolar; for rifampicin 5 micromolar, 10 micromolar and 25 micromolar; and for mevastatin 10 micromolar, 50 micromolar and 100 micromolar. Results are expressed as fold increase in luciferase activity above DMSO-treated cells and are the mean +/− standard deviation of six determinations. The lowest dose of each drug is represented as increasing from left to right.

FIG. 17 depicts the effects of plating stable cell lines in 24 and 96 well plates. 101L cells were plated in 24 or 96 well plates and exposed to various doses of the Ah receptor ligand benzanthracene. After 18 hour exposures, luciferase activity was assessed. Results are expressed as the mean of three different experiments +/− SD.

FIG. 18 depicts a time response curve of various CYP1A1 inducers. The maximal time period for inducer exposure was determined by establishing a time course of inducer mediated luciferase activity in 101 L cells and with the 96 well plates. Enhanced activity was observed within 6 hours of dosing with benzanthracene (100 micromolar), omeprazole (100 micromolar) and 3-methylcholanthrene (10 micromolar). Cells were also treated with rifampincin (100 micromolar) as a negative control. Each point represents the mean of results from three experiments +/− SD.

FIG. 19 depicts dose response curve of various known CYP1A1 inducers. The effects of various CYP1A1 inducers were determined using 96 well plate format and the 101L cells. Dose response curves were generated to TCDD (0.5 to 2.2 nanomolar (panel A), benzanthracene and omeprazole (1 to 200 micromolar) (panel B). Each point represents the mean of results from three experiments +/− SD.

FIG. 20 depicts dose response curves for various flavonoids. Using the 96 well plate format and the 101L cells, dose response curves were generated for GTE (inset). Doses ranged from 0.01 milligrams/ml to 0.2 milligrams/ml and 18 hours of exposure. Dose response curves were also determined for EGCG, quercetin, curcumin, kaempferol, naringenin, apigenin, and resveratrol and ranged from 1 to 20 micromolar. Exposure to each agent was for 18 hours. Each point represents the mean of results from three experiments +/− SD.

FIG. 21 depicts the effects of co-treatment with TCDD and each flavonoid. The CYP1A1 containing cell line was treated with 10 micromolar of each flavonoid or 0.1 milligrams/ml of GTE and 2 nanomoles of TCDD. Cells were exposed to both agents for 18 hours. Results represent the mean of three experiments +/− SD.

### SUMMARY

The in vitro system described herein can detect induction of drug metabolizing enyymes, including P450s such as CYP3A4. The disclosed methods can detect transcriptional activation by xenobiotics of an appropriate enhancer and reporter gene that have been optionally independently stably transfected into a host cell, such as human hepatoma cells. The system can be utilized in a microtiter plate format and results can optionally be obtained with an appropriate microtiter plate reader within two or three days of drug candidate application to the cells. The advantages of this in vitro transcription system as compared to isolated human hepatocytes or liver slices are numerous, including increased consistency and reproducibility of the assay. Also, inter-individual or inter-sample variability and culture conditions that can influence the results of an assay are addressed using the systems of the present invention. The present system can be formatted for high throughput assays and can predict a two-fold or greater induction of a specific drug metabolizing protein encoding gene in a relatively short time period.

In one preferred aspect of the present invention, the in vitro system is high throughput in nature and can assess CYP3A4 induction. This preferred aspect of the present invention includes the regulatory region of the CYP3A4 gene named the PXRE and the transcription factor PXR. The PXRE is operably linked to a reporter gene such as luciferase, such as on a plasmid. The plasmid containing the PXRE and reporter gene is then stably transformed into a hepatoma cell line, such as HepG2. Once transformed, the PXR can bind to the PXRE and activate transcription. This can occur when the PXR is stimulated by an appropriate ligand, such as a drug. A nucleic acid molecule encoding a drug metabolizing protein other than CYP3A4 or other than P450's can be used by substituting nucleic acid molecules. Appropriate regulatory regions other than PXRE can also be used, such that the regulatory region is appropriate for the nucleic acid molecule encoding a drug metabolizing enzyme or transporter. In addition, reporter genes other than luciferase, such as detectable proteins, such as Green Fluorescent Protein (GFP) or its variations, or other enzymes, such as beta-galactosidase, beta-lactamase or alkaline phosphatase can be used in this system. Alternative cells can be used, but cells that are derived from tissues involved in drug metabolism are preferred.

The present invention recognizes that cell based systems for evaluating compound interactions can be made using appropriate nucleic acid molecules that include one or more enhancers or promoters for a nucleic acid molecule encoding a protein involved in drug metabolism operably linked to a nucleic acid molecule encoding a reporter gene, and a nucleic acid molecule encoding an intracellular receptor or transcription factor. These nucleic acid molecules can be extra chromosomally or stably integrated into the genome of a cell. In addition, in certain cases the nucleic acid molecules can be endogenous to the chromosome of the cell, particularly in the case where the nucleic acid molecule encodes an intracellular receptor, transporter or transcription factor.

One aspect of the present invention provides a cell that includes a first nucleic acid molecule that includes: a promoter or enhancer operable for a nucleic acid molecule encoding a protein involved in drug metabolism and a reporter gene. Preferably, the promoter or enhancer is operably linked to said reporter gene. The cell also includes a second nucleic acid encoding an intracellular receptor or transcription factor, such that when the intracellular receptor or transcription factor is bound or activated with a compound, said intracellular receptor or transcription factor can operably bind with said promoter or enhancer resulting in the expression of said reporter gene. When the cell is contacted with a compound that induces the expression of the protein involved in drug metabolism, the reporter gene is expressed.

A second aspect of the present invention provides a method for evaluating compounds for the property of inducing the expression of a gene encoding a protein involved in drug metabolism, including; providing a test compound; contacting the test compound with a cell of the present invention; and detecting the expression of said reporter gene. The expression of the reporter gene is indicative that said compound altered the expression of a gene encoding a protein involved in drug metabolism.

### DETAILED DESCRIPTION OF THE INVENTION

Definitions

Unless defined otherwise, all technical and scientific terms used herein have the same meaning as commonly understood by one of ordinary skill in the art to which this invention belongs. Generally, the nomenclature used herein and the laboratory procedures in cell culture, chemistry, microbiology, molecular biology, cell science and cell culture described below are well known and commonly employed in the art. Conventional methods are used for these procedures, such as those provided in the art and various general references (Sambrook et al., Molecular Cloning: A Laboratory Manual, 2nd edition, Cold Spring Harbor Press, Cold Spring Harbor, N.Y. (1989); Ausubel et al., Current Protocols in Molecular Biology, John Wiley and Sons (1998); Harlowe and Lane, Antibodies, a Laboratory Manual, Cold Spring Harbor Press (1988)). Where a term is provided in the singular, the inventors also contemplate the plural of that term. The nomenclature used herein and the laboratory procedures described below are those well known and commonly employed in the art. As employed throughout the disclosure, the following terms, unless otherwise indicated, shall be understood to have the following meanings:

A “nucleic acid molecule” is a polynucleotide. A nucleic acid molecule can be DNA, RNA, or a combination of both. A nucleic acid molecule can also include sugars other than ribose and deoxyribose incorporated into the backbone, and thus can be other than DNA or RNA.

A nucleic acid can comprise nucleobases that are naturally occurring or that do not occur in nature, such as xanthine, derivatives of nucleobases such as 2-aminoadenine and the like. A nucleic acid molecule of the present invention can have linkages other than phosphodiester linkages. A nucleic acid molecule can also be a peptide nucleic acid molecule. A nucleic acid molecule can be of any length, and can be single-stranded or double-stranded, or partially single-stranded and partially double-stranded.

A “probe” or “probe nucleic acid molecule” is a nucleic acid molecule that is at least partially single-stranded, and that is at least partially complementary, or at least partially substantially complementary, to a sequence of interest. A probe can be RNA, DNA, or a combination of both RNA and DNA. It is also within the scope of the present invention to have probe nucleic acid molecules comprising nucleic acids in which the backbone sugar is other than ribose or deoxyribose. Probe nucleic acids can also be peptide nucleic acids. A probe can comprise nucleolytic-activity resistant linkages or detectable labels, and can be operably linked to other moieties, for example a peptide.

A single-stranded nucleic acid molecule is “complementary” to another single-stranded nucleic acid molecule when it can base-pair (hybridize) with all or a portion of the other nucleic acid molecule to form a double helix (double-stranded nucleic acid molecule), based on the ability of guanine (G) to base pair with cytosine (C) and adenine (A) to base pair with thymine (T) or uridine (U). For example, the nucleotide sequence 5′-TATAC-3′ is complementary to the nucleotide sequence 5′-GTATA-3′.

“Substantially complementary” refers to nucleic acids that will selectively hybridize to one another under stringent conditions.

“Selectively hybridize” refers to detectable specific binding. Polynucleotides, oligonucleotides and fragments thereof selectively hybridize to target nucleic acid strands, under hybridization and wash conditions that minimize appreciable amounts of detectable binding to nonspecific nucleic acids. High stringency conditions can be used to achieve selective hybridization conditions as known in the art. Generally, the nucleic acid sequence complementarity between the polynucleotides, oligonucleotides, and fragments thereof and a nucleic acid sequence of interest will be at least 30%, and more typically and preferably of at least 40%, 50%, 60%, 70%, 80%, 90%, and can be 100%. Conditions for hybridization such as salt concentration, temperature, detergents, and denaturing agents such as formamide can be varied to increase the stringency of hybridization, that is, the requirement for exact matches of C to base pair with G, and A to base pair with T or U, along the strand of nucleic acid.

“Corresponds to” refers to a polynucleotide sequence that shares identity (for example is identical) to all or a portion of a reference polynucleotide sequence. In contradistinction, the term “complementary to” is used herein to mean that the complementary sequence will base pair with all or a portion of a reference polynucleotide sequence. For illustration, the nucleotide sequence 5′-TATAC-3′ corresponds to a reference sequence 5′-TATAC-3′ and is complementary to a reference sequence 5′-GTATA-3′.

“Sequence identity” or “identical” means that two polynucleotide sequences are identical (for example, on a nucleotide-by-nucleotide basis) over the window of comparison. “Partial sequence identity” or “partial identity” means that a portion of the sequence of a nucleic acid molecule is identical to at least a portion of the sequence of another nucleic acid molecule.

“Substantial identity” or “substantially identical” as used herein denotes a characteristic of a polynucleotide sequence, wherein the polynucleotide comprises a sequence that has at least 30 percent sequence identity, preferably at least 50 to 60 percent sequence identity, more usually at least 60 percent sequence identity as compared to a reference sequence over a comparison window of at least 20 nucleotide positions, frequently over a window of at least 25 to 50 nucleotides, wherein the percentage of sequence identity is calculated by comparing the reference sequence to the polynucleotide sequence that may include deletions or additions which total 20 percent or less of the reference sequence over the window of comparison. “Substantial partial sequence identity” or “substantially partially identical” is used when a portion of a nucleic acid molecule is substantially identical to at least a portion of another nucleic acid molecule. As used herein “identity” or “identical” refers to the base composition of nucleic acids, and not to the composition of other components, such as the backbone that can be comprised of one or more sugars and one or more phosphates, or can have other substituted moieties.

A “mutation” is a change in the genome with respect to the standard wild-type sequence. Mutations can be deletions, insertions, or rearrangements of nucleic acid sequences at a position in the genome, or they can be single base changes at a position in the genome, referred to as “point mutations”. Mutations can be inherited, or they can occur in one or more cells during the lifespan of an individual.

“Hybridization” is the process of base-pairing of single-stranded nucleic acids, or single-stranded portions of nucleic acids, to create double-stranded nucleic acids or double-stranded portions of nucleic acid molecules.

A “single nucleotide polymorphism” or “SNP” is a position in a nucleic acid sequence that differs in base composition in nucleic acids isolated from different individuals of the same species.

“Operably linked” refers to a juxtaposition wherein the components so described are in a relationship permitting them to function in their intended manner. For example, a control sequence such as promoter or enhancer or other regulatory sequence operably linked to a coding sequence is positioned in such a way that expression of the coding sequence is achieved under conditions compatible with control sequences.

“Operable” in the sense of a control sequence being operable for a nucleic acid molecule encoding a polypeptide or protein, such as a protein involved in drug metabolism, refers to the ability of the control sequence to regulate the expression of such polypeptide or protein under appropriate configurations, such as being operably linked and under appropriate conditions, such as binding of appropriate modulators in appropriate configurations to the control sequence.

A “promoter” refers to a nucleic acid molecule, such as in DNA, to which RNA polymerase binds to begin transcription. A promoter can be considered a component of the gene control region where the transcription factors and the polymerase assemble to control transcription.

An “enhancer” refers to regulatory nucleic acid molecules, such as DNA sequences, to which gene regulatory proteins bind, which can influence the rate of trancription of a structural gene. Examples of enhancers include GAL4 protein attaching to a regulatory region of the LacZ gene to influence expression of beta-galactosidase. Another example is the pregnane X receptor (PXR) which binds to a DNA sequence termed PXRE or XREM that regulates the rate of transcription of the enzyme CYP3A4. A further example is the Ah receptor (AhR), which binds to specific DNA sequences termed DRE, the dioxin response element, in the regulatory region of CYPs 1A1 and 1A2 to regulate the rate of transcription.

A “protein involved in drug metabolism” refers to a protein or polypeptide, such as a protein, that is capable of metabolism or modulating the metabolism of a xenobiotic such as a drug. Such modulating includes changing the chemical structure of the xenobiotic through catalytic reactions and covalent or non-covalent bonds, altering the permeability of a xenobiotic into or out of a cell, or transporting a xenobiotic into or out of a cell.

A “drug metabolizing enzyme” refers to enzyme proteins that catalyze the covalent modification of xenobiotics such as drugs that are foreign to the host. Such covalent modifications can be any, but are preferably oxidation or conjugation reactions. The oxidation reactions generally result in water soluble metabolites or metabolites with increased water solubility. For example CYP3A4 metabolizes the drug erythromycin to a demethylated metabolite, increasing its polarity. Glucuronosyltransferase 1 (UGT1) adds a glucuronide to acetaminophen to increase its polarity. CYP2C 19 metabolizes S-mephenytoin by adding an hydroxyl group to the anticonvulsant. Generally, by increasing the polarity of the xenobiotic, the modified xenobiotic is more readily eliminated from the subject, such as through the urine.

A “reporter gene” refers to a region of a nucleic acid molecule such as DNA that encodes a protein that is readily detected by an assay. This region can replace the normal coding region of a gene. For example, the luciferase gene encodes the luciferase protein that can produce luminescent products can be detected by a luminometer. The LacZ gene encodes the beta-galactosidase protein that can convert certain substrates to colored forms that can be detected colormetrically or fluorimetrically in the presence of an appropriate enzymatic substrate. Chloramphenical acetyl transferase (CAT) is an enzyme that metabolizes chloramphenicol and results of this reaction can be visualized by a radiometic TLC assay.

An “intracellular receptor” refers to a polypeptide or protein residing within a cell that binds a molecule, including extracellular signaling molecules, such as ligands, and initiates a response in the cell. Examples of intracellular receptors include the Ah receptor or PXR.

A “hormone receptor” refers to steroid hormone receptors that bind to hormones that diffuse into the cell across the plasma membrane. Steroid receptors such as the receptor for thyroid hormone or vitamin D bind their ligand and then bind to specific DNA sequences within the genes that the ligand regulates. Examples include the estrogen receptor, the progesterone receptor or cortisol receptor.

A “transporter” refers to proteins within the plasma membrane that carry or otherwise direct molecules across a cell membrane. Transporters can be specific transporters for specific ligands, general transporters for a group of ligands, active transporters that utilize energy such as ATP or the electron motive force, or passive transporters that do not utilize energy of the cell.

Molecules can be transported into or out of a cell depending on the transporter and the conditions that it is under. Examples include the sodium-potassium ATPases and P-glycoprotein (MDR1) that transports drug metabolites from inside the cell to outside the cell.

A “transcription factor” refers to any polypeptide or protein that can initiate or regulate transcription in a cell, such as but not limited to a eukaryotic cell. These include gene regulatory proteins that bind to enhancers and the general transcription factors that do not act in such a specific manner. Examples of transcription factors include TFIID, a general transcription factor, or a specific receptor such as PXR. HNF1 is another transcription factor that regulates expression of genes in a tissue specific manner.

To be “bound” in the sense of a polypeptide such as an, intracellular receptor, transporter or transcription factor being bound with a compound, refers to these elements being in contact such that if the polypeptide and compound are bound, then the activity of the resulting complex is different from the activity of the individual elements.

To “operably bind” is to have one element bound to another element, wherein the resulting complex can perform a function. For example, a polypeptide can bind a compound and the resulting complex can operably bind with a control sequence to modulate expression of a gene operably linked to such control sequence.

A “compound” refers to any chemical, test chemical, drug, new chemical entity (NCE) or other moiety. For example, a compound can be any foreign chemical (xenobiotic) not normally present in a subject such as mammals including humans. A compound can also be an endogenous chemical that is normally present and synthesized in biological systems, such as mammals including humans. In one aspect, oxidation of compounds by enzymes generally results in a more water-soluble, easily excretable product. Examples include food additives, steroid hormones and drugs.

To “induce” refers to an increase in expression of a polypeptide such as an enzyme, such as enzymes involved in drug metabolism, in the presence of a compound relative to the amount of expression of such polypeptide in the absence of the compound. For example, a compound, such as a test compound, such as a drug, can induce the expression of a P450 enzyme, such that the amount of P450 enzyme produced in the presence of the compound is greater than the amount of P450 enzyme produced in the absence of the compound.

A “P450” refers to a member of a super-family of heme containing monooxygenases involved in the catalytic oxidation of xenobiotics such as drugs and endobiotics including steroid hormones. Examples include but are not limited to CYP2C9, CYP3A4 and CYP1A2.

A “glucuronyl transferase” or “UGTs” refers to polypeptides and proteins involved in glucuronidation, a major pathway that enhances the elimination of many lipophilic xenobiotics and endobiotics to more water-soluble compounds. The UDP-glucuronosyltransferase family catalyzes the glucuronidation of the glycosyl group of a nucleotide sugar to an acceptor compound at a nucleophilic functional group of oxygen, nitrogen, sulphur, and carbon with the formation of a beta-D-glucuronide product. There are over thirty five known different UGT gene products that have been divided into two subfamilies, UGT1 and UGT2, based on sequence identities. Examples include UGT1A2, UGT2B7 and UGT1A8.

A “glutathione transferase” refers to enzymes that are soluble proteins predominantly found in the cytosol of hepatocytes. These enzymes catalyze the conjugation of a variety of compounds with the endogenous tripeptide, glutathione. Cytosolic glutathione S-transferases can be divided into four families, termed alpha, mu, pi and theta, each having different but sometimes overlapping substrate specificities. There are also microsomal glutathione transferases residing, for example, in the endoplasmic reticulum (ER). Examples include but are not limited to GST(mu) and GST(alpha).

A “sulfo transferase” refers to polypeptides or proteins such as enzymes that catalyze the sulfation of structurally diverse xenobiotics including drugs and endogenous compounds. These reactions involve the transfer of a sulfuryl group from 3′-phophoadenosine 5′-phophosulfate (PAPS) to the hydroxyl/amino groups of acceptor molecules forming sulfuric acid esters and sulfamates. Sulfate conjugation generally results in a detoxification producing water soluble metabolites. Sulfation is also an important factor in the regulation of steroid biosynthesis and inactivation and excretion of endogenous hormones. Several isoforms of these enzymes are present in humans. Examples include but are not limited to hHST, hP-PST and hM-PST.

An “N-Acetyltransferase” refers to proteins or polypeptides such as enzymes that conjugate arylamines with an acetyl group. There are distinct genes within this family of enzymes. For example, NAT1 and NAT2 that encode for N-acetyltransferase activities in humans. NAT1 activity is monomorphically distributed in human tissues, whereas NAT2 5 exhibits a polymorphism that allows the detection of phenotypically slow and rapid acetylators. N-acetylation of arylamines represents a competing pathway for N-oxidation, a metabolic activation step occurring in the liver. Heterocyclic amines are activated by acetylation by the NAT2 transferases.

A “P-glycoprotein” or “Pgp” refers to a product of the MDR1 gene. Its function is to transport drugs and steroids across a cell membrane. Pgp may be a determinant of the magnitude of CYP3A induction. Pgp may influence PXR ligand interaction and the CYP3A inductive response to steroids and xenobiotics.

An “enzyme” refers to a polypeptide having a catalytic activity. Detectable enzymes are enzymes that when acting upon an appropriate substrate will produce a detectable product. The detectable product is preferably detected optically, such as via the emission of light, such as fluorescence, luminescence or chemiluminescence, or by color, such as by the formation of a chromogen. Preferred detectable enzymes include, but are not limited to beta-lactamase, luciferase and beta-galactosidase.

A “detectable protein” is a polypeptide that has a physical property that is detectable. Preferred detectable proteins are proteins that are inherently fluorescent, such as Green Fluorescent Protein (GFP), SPAP renillin fluorescent protein and their derivatives.

An “extra chromosomal element” refers to a nucleic acid molecule that when present within a cell is non integrated within the genome of such cell. Examples of extra chromosomal elements include plasmids and Yeast Artificial Chromosomes (YACs).

“Within the chromosome” of a cell in the context of a nucleic acid molecule refers to a nucleic acid molecule is within the chromosome or genome of the cell as opposed to being an extra chromosomal element. A nucleic acid molecule within the chromosome of a cell can be “inserted” within the genome, such as by homologous recombination or other methods, or can be “endogenous to the chromosome.” In the case of endogenous to the chromosome, the nucleic acid molecule is within the chromosome at its original locus.

To “directly” produce an event or form a structure is to not have intermediary steps or structures. For example, A and B forming AB directly interact because there is no structure between A and B. Also, C and D reacting to form E directly interact to form E because there are no intermediary steps between the reaction of C and D to form E.

To “indirectly” produce an event or form a structure is to have an intermediate step or structure. For example, A and B forming ABC have A and B indirectly interacting because there is a structure between A and B. Also, E and F forming G which reacts with H to form I is an indirect formation of I from E and F because an intermediary step is involved in the process of making I.

A “cell” is any cell, such as a prokaryotic or eukaryotic cell. A cell is preferably a eukaryotic cell and is preferably from a multi-cellular organism, but can be a unicellular organism such as a yeast or other free-living eukaryotics. A cell can be obtained from an organism, such as an animal or a human, and provided in primary culture or continuous cultures such as in the case of a cell line. A cell can be part of a population of cells, such as a population of similar cells, such as cells from the same tissue or organ, or of substantially the same cells, such as in a clonal population of cells. The cells can be obtained from any appropriate organism, such as through routine sampling, such as through biopsy for the collection of tissues or through the collection fluids, such as blood, using routine methods. Cells are preferably mammalian cells and are preferably human cells, but that need not be the case. Cells are also preferably derived from a tissue that naturally exhibit relatively high levels of expression of enzymes that are involved in drug metabolism, such as, but not limited to, liver, intestine, lung or kidney. Cells can also be transformed cells, which are cells that have been genetically altered by genetic engineering processes, such as by the introduction of extra chromosomal elements or integration of nucleic acid molecules into the chromosome of the cell.

“High throughput screening” refers to methods for screening for activity of compounds, such as test compounds such as drugs, takes place at a rate of between about 5 assays or samples per day and about 10,000 assays or samples per day, preferably between about 10 assays or samples per day and about 1,000 assays or samples per day and more preferably between about 15 and about 500 assays or samples per day.

Introduction

The present invention provides improved cells and methods for identifying compounds that alter protein expression, such as chemicals or drugs. The invention provides other benefits as well.

As a non-limiting introduction to the breadth of the present invention, the present invention includes several general and useful aspects, including:

1) a cell that includes a first nucleic acid molecule that includes: a promoter or enhancer operable for a nucleic acid molecule encoding a protein involved in drug metabolism and a reporter gene; and a second nucleic acid encoding an intracellular receptor or transcription factor; so that when the intracellular receptor or transcription factor is in contact with a compound, or directly or indirectly activated by a compound or directly or indirectly modulated by a compound, the intracellular receptor or transcription factor can operably bind with the promoter or enhancer resulting in the expression of said reporter gene; and

2) a method for evaluating compounds for the property of inducing the expression of a gene encoding a protein involved in drug metabolism, including; providing a test compound; contacting the test compound with a cell of the present invention; and detecting the expression of said reporter gene.

These aspects of the invention, as well as others described herein, can be achieved using the methods, articles of manufacture, and compositions of the present invention. To gain a full appreciation of the scope of the present invention, it will be further recognized that various aspects of the present invention can be combined to make desirable embodiments of the invention.

I. A Cell for Evaluating Enhanced Protein Expression by Test Compounds

The present invention includes a cell that includes a first nucleic acid molecule that includes: a promoter or enhancer operable for a nucleic acid molecule encoding a protein involved in drug metabolism and a reporter gene; and a second nucleic acid encoding an intracellular receptor or transcription factor; so that when the intracellular receptor or transcription factor is bound with a compound, the intracellular receptor or transcription factor can operably bind with the promoter or enhancer resulting in the expression of the reporter gene.

First Nucleic Acid Molecule

The cell includes a first nucleic acid molecule that includes a promoter or enhancer operable for a nucleic acid molecule encoding a protein involved in drug metabolism, including enzymes and transporters, and a reporter gene. The promoter or enhancer is operably linked to the reporter gene. In this way, when the promoter or enhancer is activated (such as by binding of a receptor/compound complex), the reporter gene is expressed. If the reporter gene is expressed at or above a detectable level, then the activation of the promoter or enhancer is reported.

The first nucleic acid molecule is preferably double stranded DNA, but that need not be the case. The first nucleic acid molecule can be extra chromosomal or be within the chromosome of the cell. Extra chromosomal elements include, but are not limited to, vectors, viruses, plasmids, YACs and linear nucleic acid molecules. Methods for preparing such plasmids, YACs and linear nucleic acid molecules that have the characteristics of the first nucleic acid molecule, such as the promoter or enhancer operably linked to the reporter gene, are known in the art. For example, nucleic acid molecules that encode promoters or enhancers operable for a nucleic acid molecule encoding a protein involved in drug metabolism are known in the art, are often times commercially available and can be prepared and cloned using routine methodologies including PCR, restriction enzymes, digestion and chemical synthesis. These promoters or enhancers can be operably linked to a reporter gene using routine methods such that when the promoter or enhancer is activated, the reporter gene is expressed. This construct can then be cloned into an appropriate vector, such as but not limited to plasmids, viral vectors, YACs and linear nucleic acid molecules. These vectors can then be used to transform a cell or population of cells. Such transformations are known in the art, such as electroporation, viral infectivity, microbalistics or passive uptake of nucleic acid molecules by cells.

If the first nucleic acid molecule includes a gene that encodes a selectable marker operably linked to a promoter, such as a constitutive promoter such as CMV promoter, MMTV promoter or SV40 promoter, cells that have taken up the nucleic acid molecule and the nucleic acid molecule is operable can be selected for. Preferred selectable markers include antibiotic resistance, such that cells that have an operable first nucleic acid molecule would be resistant to a particular antibiotic whereas cells that do not have such a first nucleic acid molecule would be susceptible to such antibiotic. In that way, cells having a first nucleic acid molecule that expresses the selectable marker can be selected and enriched. Alternative selectable markers include fluorescent proteins, such as Green Fluorescent Protein (GFP) or its derivatives, or enzymes that catalyze the formation or transformation of fluorescent substrates or products, such as beta-lactamase. Under these conditions, fluorescence activated cell sorting (FACS) can be used to isolate cells having a desired fluorescent property.

The first nucleic acid molecule can be extra chromosomal, or can be integrated within the genome of the cell. When the first nucleic acid is integrated within the genome of the cell, the first nucleic acid becomes stably integrated, which results in a cell having greater reliability and reproducibility than transiently transfected cells. Certain vectors, such as viral vectors, particularly retroviral vectors, can integrate within the genome. Also, homologous recombination can be used to promote the insertion of a nucleic acid molecule within the genome of a cell using methods, such as those described in U.S. Pat. No. 6,187,305 to Treco et al., issued Feb. 13, 2001 and U.S. Pat. No. 6,063,630 to Treco et al., issued May 16, 2000. In the alternative, transformed nucleic acid molecules can spontaneously integrate into a host genome. The integration of a first nucleic acid molecule within the genome of a cell can be monitored by screening cells for the loss of a selectable marker or reporter gene because transiently transfected cell lines tend to eject nucleic acid molecules that are not integrated into the genome of the cell. Thus, the selectable marker or reporter gene would tend to be lost over time, such as through repeated passages of cell lines.

In one aspect of the present invention, the reporter gene is endogenous to the chromosome of the cell. In this instance, the reporter gene preferably encodes an enzyme that can readily be determined, such as by detectable enzymatic substrates or products thereof. In this instance, a nucleic acid molecule that includes a promoter or enhancer operable for the desired reporter gene is engineered into a vector such that the integration of that vector is directed to a locus in the genome at or near the reporter gene. Integration of the nucleic acid construct that includes the promoter or enhancer can be directed using homologous recombination methodologies as they are known in the art, such as those described in U.S. Pat. No. 6,187,305 to Treco et al., issued Feb. 13, 2001 and U.S. Pat. No. 6,063,630 to Treco et al., issued May 16, 2000. Also, spontaneous or non-directed recombination methodologies can be used as they are known in the art. Not all such homologous recombination events will result in an operable link between the promoter or enhancer and the reporter gene, thus the cell or population of cells should be screened for such operable link. For example, if the event does result in an operable link, activation of the enhancer or promoter would result in the expression of the reporter gene. Such expression can be monitored and screened using appropriate detectable enzymatic substrates and/or products.

In another aspect of the present invention, the enhancer or promoter is endogenous to the chromosome of the cell. In this instance, a nucleic acid molecule that includes a reporter gene operable for the enhancer or promoter is engineered into a vector such that the integration of that vector is directed to a locus of the genome at or near the promoter or enhancer. Integration of the nucleic acid construct that includes the reporter gene can be directed using homologous recombination methodologies as they are known in the art, such as those described in U.S. Pat. No. 6,187,305 to Treco et al., issued Feb. 13, 2001 and U.S. Patent No. 6,063,630 to Treco et al., issued May 16, 2000. Not all such homologous recombination events will result in an operable link between the promoter or enhancer and the reporter gene, thus the cell or population of cells should be screened for such operable link. For example, if the event does result in an operable link, activation of the enhancer or promoter would result in the expression of the reporter gene. Such expression can be monitored and screened using appropriate detectable enzymatic substrates and/or products.

Second Nucleic Acid Molecule

The cell also includes a second nucleic acid encoding an intracellular receptor or transcription factor. When the intracellular receptor or transcription factor is bound with a compound, the intracellular receptor or transcription factor can operably bind with the promoter or enhancer resulting in the expression of the reporter gene.

The second nucleic acid molecule is preferably double stranded DNA, but that need not be the case. The second nucleic acid molecule can be extra chromosomal or be within the chromosome of the cell. Extra chromosomal elements include, but are not limited to, vectors, viruses, plasmids, YACs and linear nucleic acid molecules. Methods for preparing such plasmids, YACs and linear nucleic acid molecules that have the characteristics of the second nucleic acid molecule, such as including a nucleic acid molecule encoding an intracellular receptor or transcription factor, are known in the art. For example, nucleic acid molecules that encode an intracellular receptor or transcription factor are known in the art, are often times commercially available and can be cloned using routine methodologies. This construct can then be cloned into an appropriate vector, such as but not limited to plasmids, viral vectors, YACs and linear nucleic acid molecules. These vectors can then be used to transform a cell or population of cells. Such transformations are known in the art, such as electroporation, viral infectivity, microbalistics or passive uptake of nucleic acid molecules by cells.

Preferably, the second nucleic acid molecule includes a regulatory element, such as a promoter or enhancer, operably linked with said nucleic acid molecule encoding an intracellular receptor or transcription factor. The regulatory element is preferably a promoter or constitutive promoter, such as SV40 promoter, MMTV promoter or CMV promoter. As discussed for the first nucleic acid molecule, there are art recognized methods to make constructs such as vectors having this type of configuration.

If the second nucleic acid molecule includes a gene that encodes a selectable marker operably linked to a promoter, such as a constitutive promoter such as CMV promoter, MMTV promoter or SV40 promoter, cells that have taken up the nucleic acid molecule can be selected for. Preferred selectable markers include antibiotic resistance, such that cells that have an operable second nucleic acid molecule would be resistant to a particular antibiotic whereas cells that do not have such a second nucleic acid molecule would be susceptible to such antibiotic. In that way, cells having a second nucleic acid molecule that expresses the selectable marker can be selected and enriched. Alternative selectable markers include reporter proteins encoded by reporter genes such as fluorescent proteins, such as Green Fluorescent Protein (GFP) or its derivatives, or enzymes that catalyze the formation or transformation of fluorescent substrates or products, such as beta-lactarnase. Under these conditions, fluorescence activated cell sorting (FACS) can be used to isolate cells having a desired fluorescent property.

In aspects of the invention where the first nucleic acid molecule and the second nucleic acid molecule both include selectable markers, it is preferable that these selectable markers be different, but that need not be the case. Different selectable markers allows the independent monitoring of both the first nucleic acid molecule and the second nucleic acid molecule in the cell.

The second nucleic acid molecule can be extra chromosomal, or can be integrated within the genome of the cell. When the second nucleic acid is integrated within the genome of the cell, the second nucleic acid becomes stably integrated, which results in a cell having greater reliability and reproducibility than transiently trasfected cells. Certain vectors, such as viral vectors, particularly retroviral vectors, can integrate within the genome. Also, homologous recombination can be used to promote the insertion of a nucleic acid molecule within the genome of a cell using methods such as those described in U.S. Pat. No. 6,187,305 to Treco et al., issued Feb. 13, 2001 and U.S. Pat. No. 6,063,630 to Treco et al., issued May 16, 2000. In the alternative, transformed nucleic acid molecules can spontaneously integrate into a host genome.

The integration of a second nucleic acid molecule within the genome of a cell can be monitored by screening cells for the loss of a selectable marker or reporter gene because transiently trasfected cell lines tend to eject nucleic acid molecules that are not integrated into the genome of the cell. Thus, the selectable marker or reporter gene would tend to be lost over time. Materials and methods for integrating nucleic acid molecules within the chromosome are known in the art (see, for example, WO 98/13353, published Apr. 2, 1998, naming Whitney et al. as inventors; WO 94/24301, published Oct. 27, 1994 to The University of Edinburgh; U.S. Pat. No. 6,187,305 to Treco et al., issued Feb. 13, 2001 and U.S. Pat. No. 6,063,630 to Treco et al., issued May 16, 2000.).

In one preferred aspect of the present invention, the gene encoding the intracellular receptor or transcription factor is endogenous to the chromosome of the cell. In particular, the gene encoding the intracellular receptor or transcription factor is in its native environment within the cell's genome, that is to say that its location and surrounding genome that includes cis-acting regulatory elements such as promoters or enhancers has not been purposely altered by human intervention.

In another aspect of the present invention, the gene encoding an intracellular receptor or transcription factor is endogenous to the chromosome of the cell but an exogenous regulatory sequence operable for the gene encoding an intracellular receptor or transcription factor, such as a promoter or enhancer, is integrated into the genome of the cell, preferably as to be operably linked with the endogenous gene encoding an intracellular receptor or transcription factor. Integration of the nucleic acid construct that includes the gene encoding an intracellular receptor or transcription factor can be directed using homologous recombination methodologies or spontaneous non-directed recombination methods as they are known in the art, such as those described in U.S. Pat. No. 6,187,305 to Treco et al., issued Feb. 13, 2001 and U.S. Pat. No. 6,063,630 to Treco et al., issued May 16, 2000.

Not all such homologous recombination events will result in an operable link between the regulatory element and the gene encoding an intracellular reporter or transcription factor, thus the cell or population of cells should be screened for such operable link. Such expression can be monitored and screened using methods appropriate for detecting the activity of such intracellular receptor or transcription factor. In the alternative, the construct to be integrated within the genome of the cell can include a reporter gene that is operably linked with the regulatory sequence used to modulate the expression of the intracellular receptor or transcription factor. In the alternative, a the reporter gene can be operably linked with a second regulatory sequence, which can be the same or different from the regulatory sequence used to modulate the expression of the intracellular receptor or transcription factor. In this instance, the sustained expression of the reporter gene indicates that the nucleic acid construct had operably integrated into the genome of the cell.

Interaction of First Nucleic Acid Molecule and Second Nucleic Acid Molecule

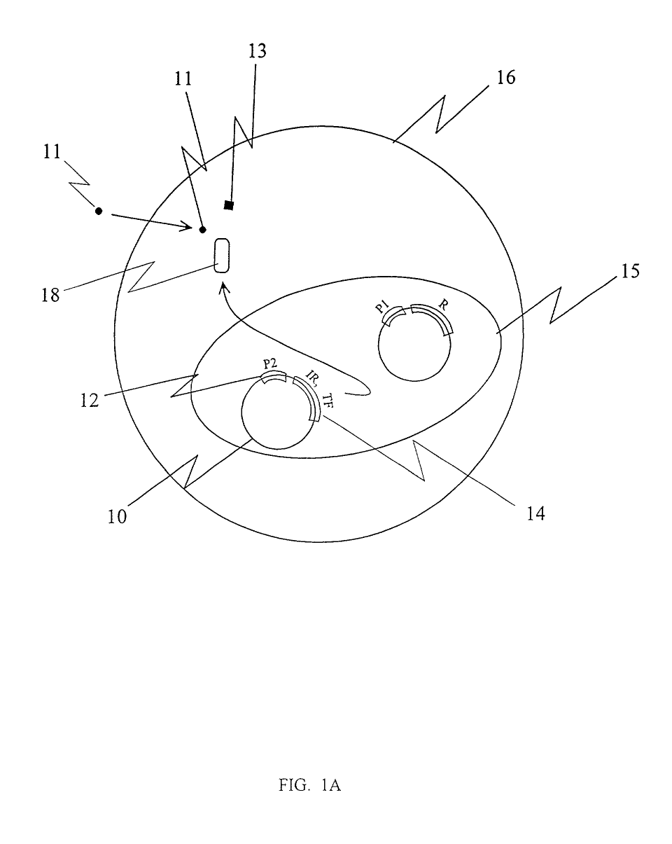

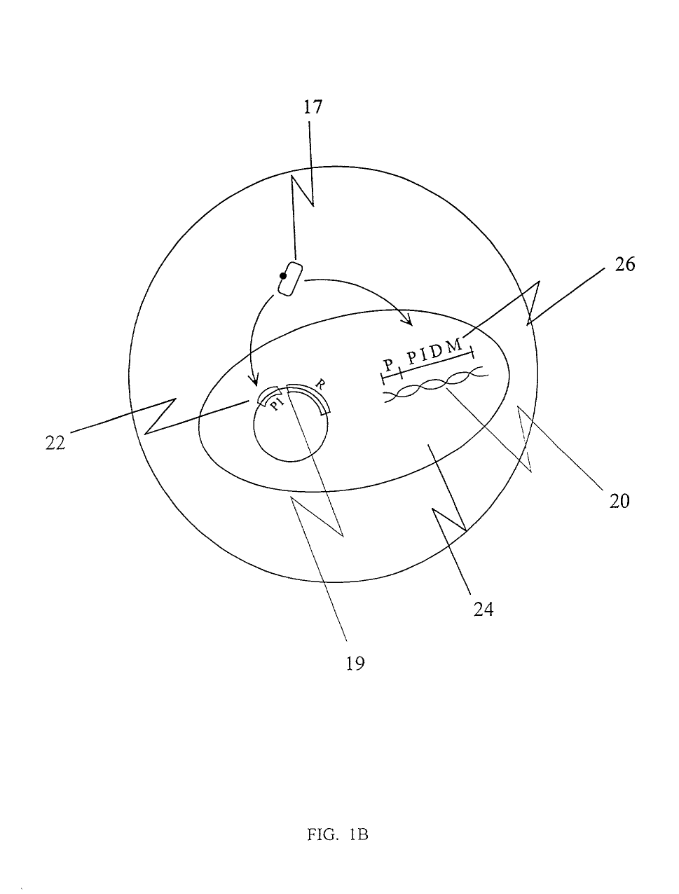

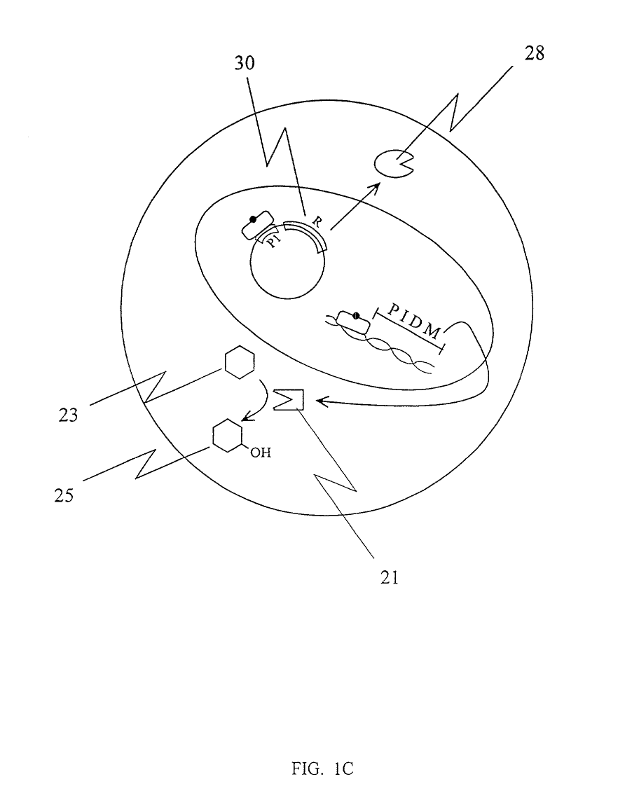

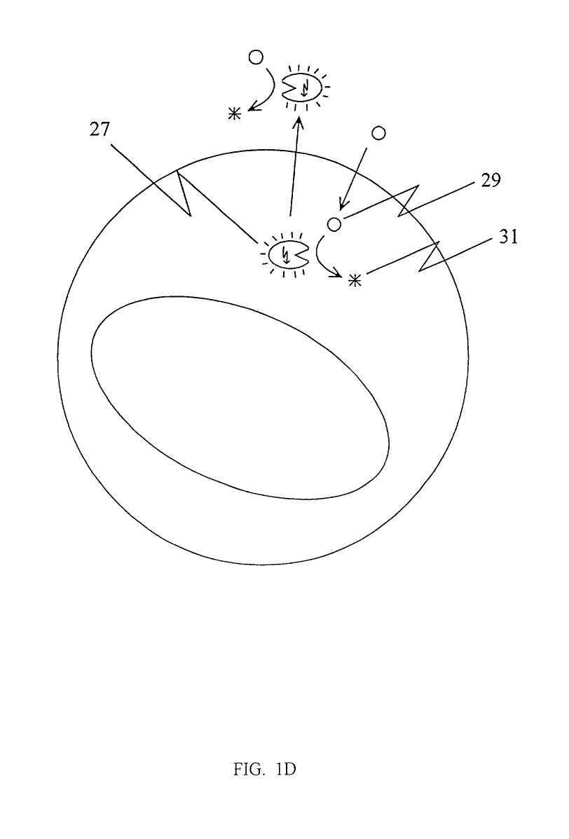

FIG. 1 through FIG. 5 depict various aspect of the present invention in diagrammatic sketches. These sketches provide the general workings of the present invention under circumstances where the first nucleic acid molecule and the second nucleic acid molecule are exogenous, endogenous, integrated or extra chromosomal. FIG. 1 depicts the general interactions of the first nucleic acid molecule and the second nucleic acid molecule. In general, when the cell is contacted with a compound that induces the expression of the enzyme or transporter involved in drug metabolism, the reporter gene is expressed. However, if the cell does not have the genes encoding the enzyme involved in drug metabolism or if such genes are not in a configuration that allows expression, the protein involved in drug metabolism may not be expressed.

As depicted in FIG. 1A, the second nucleic acid molecule (10) within a cell (16) includes a regulatory element (12) to modulate the expression of a gene encoding an intracellular receptor or transcription factor (14). The expressed intracellular receptor or transcription factor (18) can then interact with a test compound (11) by appropriate interactions, such as binding, associating, modulating and the like. The test compound can enter the cell by way of active transport or passive transport mechanisms. The test compound may optionally be modified by this transport process to form a modified test compound (13). As shown in FIG. 1B, the transcription factor or receptor can specifically bind with an appropriate test compound or metabolite if they are receptor—ligand pairs to form a complex (17). This complex (17) can bind with the first nucleic acid molecule (19) and optionally with the genome of the cell (20). When binding with the first nucleic acid molecule (19) or the genome of the cell (20), the complex (17) can bind with the regulatory element operable for a nucleic acid molecule encoding a protein involved in drug metabolism (22) or with an endogenous regulatory element (24) that can bind with such complex (17). In the latter case, the endogenous regulatory element can modulate the expression of a gene encoding a protein involved in drug metabolism (26). However, binding to the endogenous regulatory element is not a requirement of the present invention, particularly in this aspect of the present invention. As shown in FIG. 1C, the binding of the complex to the regulatory element on the first nucleic acid molecule results in the expression of a reporter (28) encoded by a reporter gene (30). The binding of the complex to the endogenous regulatory sequence can result in the expression of an endogenous protein involved in drug metabolism (21). The endogenous protein involved in drug metabolism can modify a compound (23) via a variety of mechanisms, such as by hydroxylation (25). The reporter can be detectable, such as by fluorescence of the reporter (27) or by the conversion of a substrate (29) to a detectable product (31) (FIG. 1D).

Thus, when the compound (11) or a modified compound (13) capable of binding with the intracellular receptor or transcription factor (18) and binding with a regulatory sequence for a nucleic acid molecule encoding a protein involved in drug metabolism (22), the reporter gene (30) is expressed as a reporter (28) which can be detected.

Proteins Involved in Drug Metabolism

The protein involved in drug metabolism can be any appropriate enzyme or transporter.

Preferred enzymes involved in drug metabolism include but are not limited to P450s, transporters, glucuronoyl transferases, N-acetyl transferases, glutathione transferases, p-glycoproteins and sulfo transferases. Preferred transporters include but are not limited to p-glycoprotein (MDR1). This protein transports drug metabolites out of a cell and can influence the rate of drug metabolism by a cell. P-glycoprotein expression may be altered by certain drugs (see, for example, Schuetz et al., Mol. Pharmacol. 49:311-318 (1999); Lan et al., Mol. Pharmacol. 58:863-869 (2000) and Wrighton et al., Drug Metab. Rev. 32:339-361 (2000)). Nucleic acid molecules encoding these types of proteins have been reported and can be isolated using standard methods in molecular biology (see, for example, Garattini, Drug Metab. Rev. 29:853-886 (1997); Schuetz et al., Mol. Pharmacol. 49:311-318 (1996)) and Nebert and Dieter, Pharmacology 61:124-135 (2000)).

Promoter or Enhancer

The regulatory sequences, such as promoters or enhancers, operable for a nucleic acid molecule encoding a protein involved in drug metabolism is preferably a promoter or enhancer for P450s, glucuronyl transferases, glutathione transferases and sulfo transferases or p-glycoprotein. Sequences of such regulatory sequences are known in the art and can be isolated using standard methods in molecular biology (see, for example, Nelson et al., DNA Cell Biol. 12:1-51 (1993); Windmill et al., Mutat. Res. 376:153-160 (1997); Schuetz et al., J. Cell Physiol. 165:261-272 (1995); Schuetz et al., Mol. Pharmacol. 49:311-318 (1996); Parker et al., J. Clin. Endocrin. And Metabol. 80:1027-1031 (1995); Brockmoller et al., Toxicol. Lett. 103:173-183 (1998); Vaury et al., Cancer Res. 55:5520-5523 (1995); Rodrigo et al., Scand. J. Gastroenterol. 34:303-307 (1999) and Munzel et al., Drug Metab. Dispos. 27:569-573 (1999)).

Reporter Gene

The reporter gene can be any appropriate reporter gene as is known in the art. A reporter gene encodes a reporter, such as a detectable protein or a detectable enzyme. Detectable proteins can be detected based on their physical characteristics, such as fluorescence in the case of fluorescent proteins such as Green Fluorescent Protein (GFP) or its derivatives. Enzymes can be detected using appropriate substrates that change properties when a protein acts on the substrate to form a product. Certain substrate—enzyme pairs can cause a change in fluorescent properties of the substrate, such as in the case of beta-lactamase acting on CCF2/AM to alter the characteristics of FRET in the CCF2/AM molecule. Fluorescence can be generated in the pair of glucuronidase activity on MUG. Chemiluminescence can be generated by activity of luminol dioxanes. Luminescence can be generated by luciferase activity on luciferin (see, for example, Alam and Cook, Anal. Biochem. 188:45-254 (1990). Colored product can be generated by beta-galactosidase activity on X-Gal substrate. The applicability of reporter genes to the study of reporter gene transcription has been discussed (Alam and Cook, Anal. Biochem. 188:45-254 (1990)).

Intracellular Receptor or Transcription Factor

In one aspect of the present invention, the intracellular receptor or transcription factor forms a complex with a xenobiotic such as a drug, chemical or metabolite thereof and directly or indirectly produces transcriptional activation of a gene encoding a protein involved in drug metabolism. This activity is depicted in the figures. In one aspect of the present invention, the intracellular receptor or transcription factor is not a hormone receptor, but that is not a requirement of the present invention. In another aspect of the present invention, the intracellular receptor or transcription factor is an orphan receptor, that is, a receptor that does not have a known or identified function. Examples of such orphan receptors include, but are not limited to, PXR and CAR (see, for example, Lehmann et al., J. Clin. Invest. 102:1016-1023 (1998); Jones et al., Mol. Endocrinol. 14:27-39 (2000); Honkakoski et al., Biochem. J. 347:321-337 (2000) and Savas et al., Mol. Pharmacol. 56:851-857 (1999)). The intracellular receptor or transcription factor can be a hormone receptor, such as but not limited to the glucocorticoid receptor.

Cells

The cells of the present invention can be any cell, including prokaryotic or eukaryotic. Cells are preferably eukaryotic and are from a mammalian subject, including a human. The cells can be of any origin, such as derived from the mesoderm, endoderm or ectoderm. The cells can be derived from any tissue, organ or fluid from a subject, but are preferably derived from the liver, kidney or lung. The cells can be provided from a subject, such as from a sample from a biopsy or autopsy, and can be primary cells such are known or can be made using methods known in the art. The cells can also be a cell line, such as are known or can be made using methods known in the art. For example, a variety of cell lines are available from the American Type Tissue Collection (see, ATCC Catalogues (2001)). The cells can also be a mixed culture such as a variety of cells or cell types are provided. For example, primary cells can include a variety of cell types, such as hepatocytes mixed with fibroblasts. Mixed cultures of different continuous cell lines or mixed cultures of primary cells and continuous cell lines can also be used. In one aspect of the present invention, the cells can be transformed such that they can express an exogenous protein or polypeptide.

In one aspect of the present invention cells can be provided from a particular subject. The identity of the subject need not be known, only that a particular subject is the source of cells. In the alternative, cells from a population of subjects, such as those having common ethnic origin or common disease states, disease conditions, physiological genotypes or phenotypes or metabolic phenotypes or genotypes can be used. These cells can be transformed to become cells of the present invention and can be used in the methods of the present invention. In this instance, the response of these cells to xenobiotics can be indicative of how that subject or population of subjects would respond metabolically and physiologically to that xenobiotic. In the case of cells from a population of subjects, cells from different subjects can be tested separately, but that need not be the case. The results of these types of studies can be collected and analyzed using bioinformatic technologies to assist in pharmacogenomic studies and methods. A variety of computer programs are available to provide such analyses, such as but not limited to statistical software that can provide linear or non-linear statistical methodologies. The selection of statistical analysis can be chosen by the skilled artisan.

The data, analysis and/or results generated using these methods is also part of the present invention. The data, analysis and/or results can be stored on appropriate information storage media, such as but not limited to magnetic media, tapes, paper or the like. The information storage media is preferably in a machine readable format, but that need not be the case. The information storage media can also be part of a machine, such as a machine having a central processing unit. Such a machine can be operating or not operating to be part of the present invention.

II. A Method for Evaluating a Test Compound for Inducing Expression of a Gene Encoding a Protein Involved in Drug Metabolism

The present invention includes a method for evaluating compounds for the property of inducing the expression of a gene encoding a protein involved in drug metabolism, including: providing a test compound, contacting the test compound with a cell of the present invention and detecting the expression of said reporter gene. The expression of the reporter gene is indicative that the test compound altered the expression of a gene encoding a protein involved in drug metabolism. The method can be in a high throughput method, but that is not a requirement of the present invention.

Various aspects of the present invention are depicted in the figures. For example, FIG. 1 depicts a series of figures for one aspect of the present invention, where the first nucleic acid molecule and second nucleic acid molecule are provided as extra chromosomal elements such as plasmids. As depicted in FIG. 1A, a regulatory element P2 modulates the transcription of the gene encoding an intracellular receptor or transcription factor. The translation product can then interact with a test compound that binds with the intracellular receptor or transcription factor. As depicted in FIG. 1B, the complex of the intracellular receptor or transcription factor and xenobiotic or test compound can then bind with the promoter or enhancer operable for a nucleic acid molecule encoding a protein involved in drug metabolism. The complex can also enter the nucleus and optionally bind with the endogenous promoter or enhancer operable for a nucleic acid molecule encoding a protein involved in drug metabolism, if present or active in such cell. Upon binding of this complex with the promoter or enhancer operable for a nucleic acid molecule encoding a protein involved in drug metabolism, a reporter gene is transcribed and translated into a reporter and optionally the endogenous enzyme involved in drug metabolism is expressed, if present or active in such cell (FIG. 1C). That reporter can be detectable by its physical properties, such as fluorescence, or can be a protein that is detectable based on its enzymatic conversion of substrate to product, such as a detectable product (FIG. 1D). In another aspect of the present invention, both the first nucleic acid molecule and the second nucleic acid molecule are provided on the same extra chromosomal element, such as a single plasmid or YAC or separate plasmids.

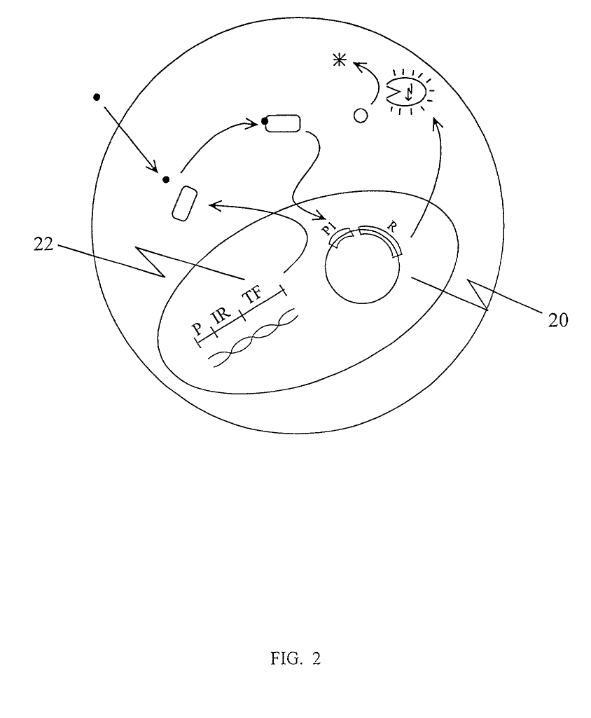

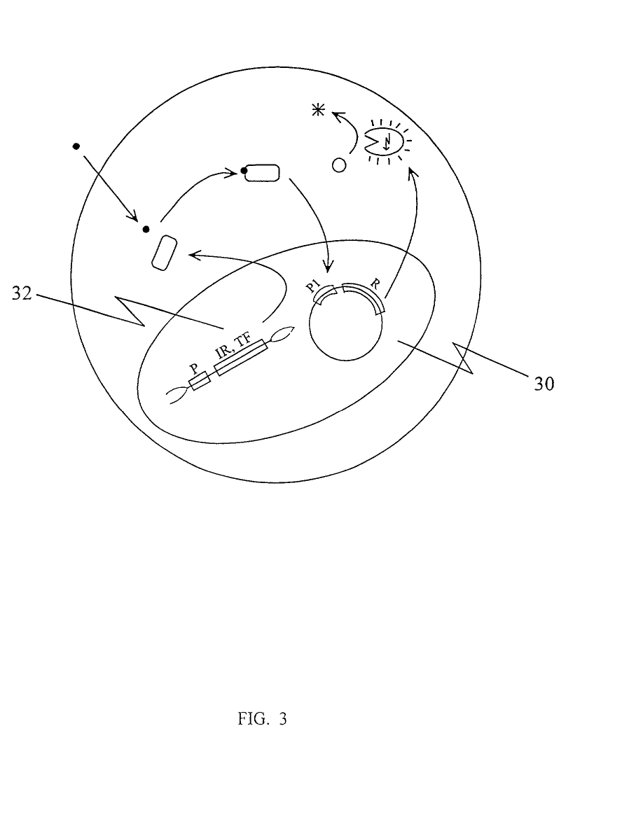

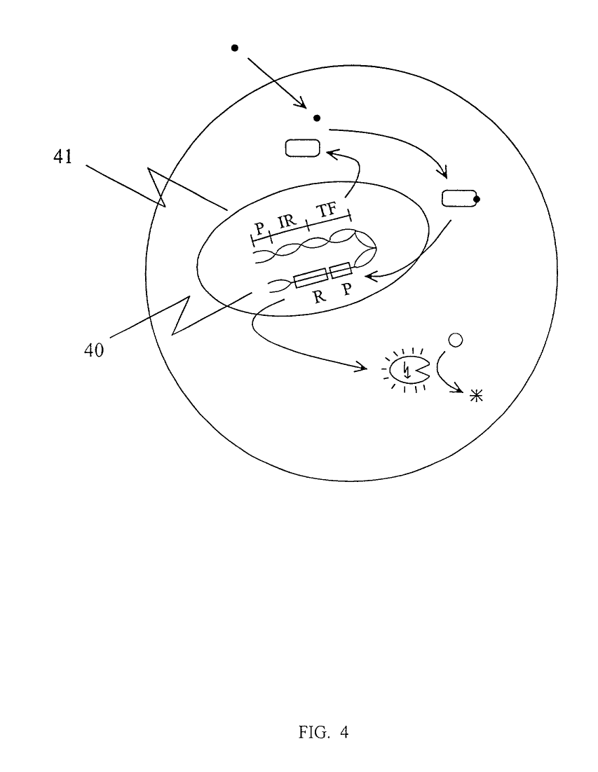

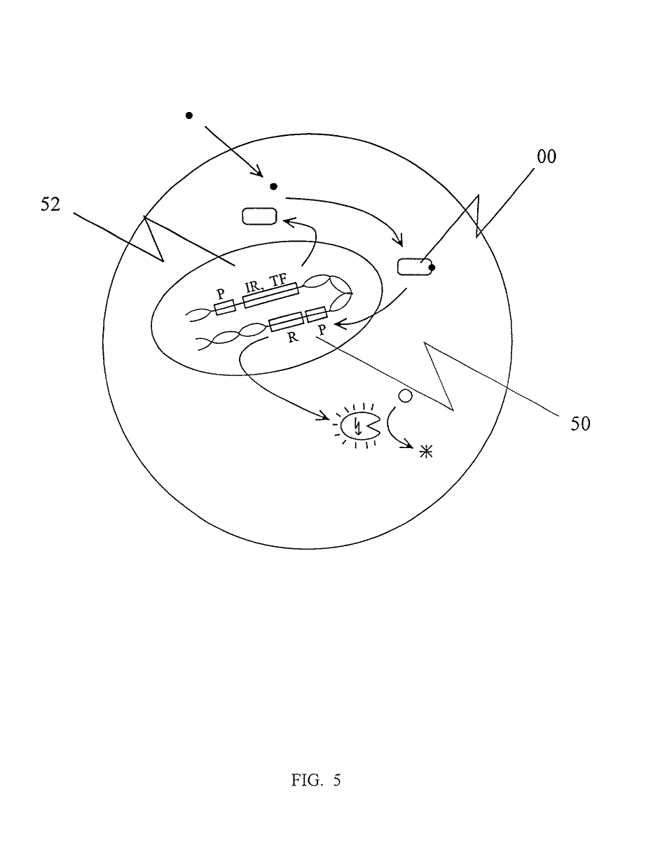

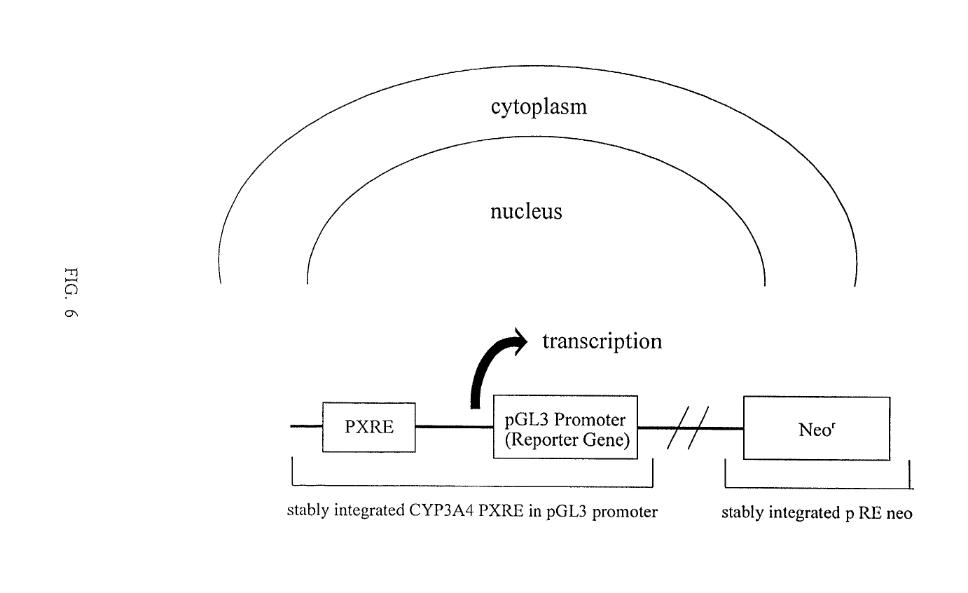

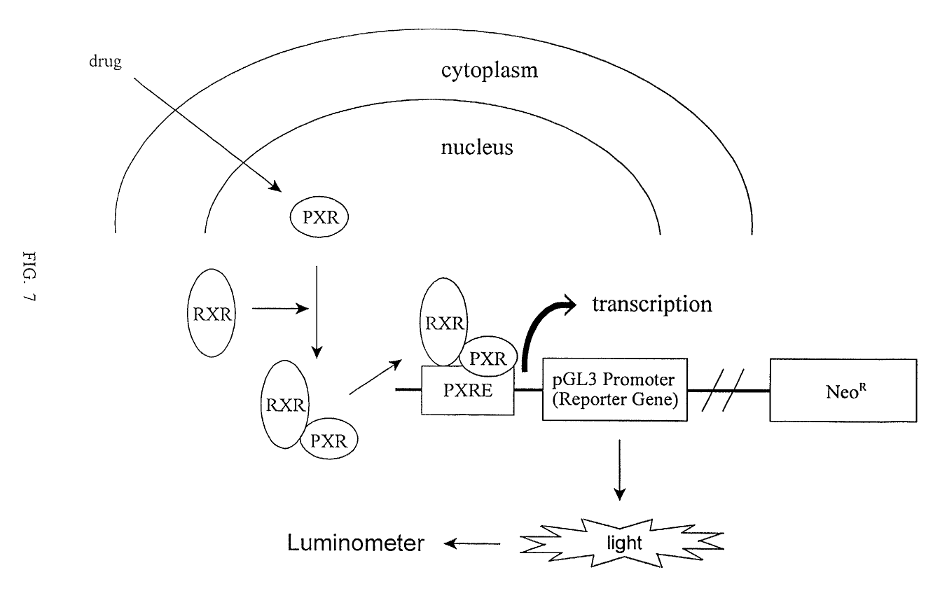

Alternatives to the aspect of the present invention depicted in FIG. 1 are also provided. For example, FIG. 2 depicts the case where the first nucleic acid molecule is an extra chromosomal element whereas the second nucleic acid molecule is endogenous to the chromosome of the cell. FIG. 3 depicts the case where the first nucleic acid molecule is an extra chromosomal element and the second nucleic acid molecule is an exogenous nucleic acid molecule integrated into the genome of the cell. FIG. 4 depicts the case where the first nucleic acid molecule is an exogenous nucleic acid molecule integrated into the genome of the cell and the second nucleic acid molecule is endogenous to the chromosome of the cell. FIG. 5 depicts the case where the first nucleic acid molecule is an exogenous nucleic acid molecule integrated into the genome of the cell and the second nucleic acid molecule is an exogenous nucleic acid molecule integrated into the genome of the cell.

The methods of the present invention can be carried out using appropriate hardware, such as tissue culture flasks or plates having appropriate surface area per flask or well. Preferably, the methods utilize appropriate plates having six, twelve, twenty-four, fourty-eight or ninety-six wells on a standard size microtiter plate footprint. The methods can also utilize plates having higher well densities, such as 192, 288, 384, 480, 576, 672, 768, 864, 960, 1056 or higher wells per plate on a standard footprint. These plates are commercially available such as through Costar and other vendors in the commercial marketplace.

The methods can be performed using human technical resources, or in part or in whole using robotics. In the later case, robotics can be used to provide high throughput capabilities that can reduce the cost and increase the reliability of the performance of the methods. Robotic systems can be made to perform these methods. For example, sample storage units known in the art can be used to store test compounds in an indexed fashion. Retrieving robotics known in the art can be used to retrieve samples from the sample storage unit for later dispensation into test vessels, such as wells of a microtiter plate, using dispensation robotics known in the art. Robotics can be used to dispense cells of the present invention and appropriate culture materials into test vessels using dispensation robotics known in the art, which can then be cultured under appropriate conditions to grow or maintain such cell cultures. Incubators, such as those known in the art, can be used to provide appropriate conditions.

Cell cultures in test vessels can be combined with test compounds using robotics, such as using dispensation robotics known in the art. The cells with test compounds can be provided appropriate conditions, such as atmosphere and temperature, for a method of the present invention, such as in an incubator as is known in the art. Reporter gene products can be detected directly, such as with detectable proteins, or with the addition of enzymatic substrates for enzymes. Enzymatic substrates can be added to test vessels using robotics, such as dispensation units. Cells can be lysed, if needed, desired or appropriate using appropriate reagents, which can be dispensed using robotic dispensation devices and methods known in the art. Detection devices known in the art, such as microtiter plate readers for chromogens, fluorescence, luminescence or the like, can be used to detect reporter gene products.

The information output or data generated using these methods can be routed to information storage devices, such as devices that include a central processing unit. The information storage device can also include information processing capabilities, such as appropriate software. This software can have the capability of making statistical comparisons or performing statistical analysis such as is known in the art, including linear and non-linear methodologies.

Such robotic systems and their components are generally known in the art and are generally described or commercially available in whole or in part from a variety of commercial vendors (see, generally WO 98/52047, published Nov. 19, 1998, naming Stylli et al. as inventors). The various steps and processes used to perform a method of the present invention can independently be performed by robotics or humans.

### EXAMPLES

### Example I

A. MATERIALS AND METHODS

Construction of Plasmids for Transfections

The full length coding region of human PXR was derived by RT-PCR from RNA obtained from a human liver sample. The forward and reverse oligonucleotide sequences were 5′-ATGGAGGTGAGACCCAAAGAA-3′(SEQ ID NO:1) and 5′-CTCAGCTACCTGTGATGCCGA-3′(SEQ ID NO:2), respectively. The PCR conditions consisted of denaturing at 94° C. for four minutes, followed by thirty cycles of 94° C. for 45 seconds, 55° C. for one minute and 72° C. for two minutes with a final extension at 72° C. for seven 15 minutes. The 1300 base pair amplified product was cloned into pCR2.1 (Invitrogen, Carlsbad, Calif.) and subjected to sequence analysis. The sequences obtained agreed over the entire coding region with that previously described (Lehmann et al., J. Clin. Invest. 102: 1016-1023 (1998)).

The cDNA was then extracted from pCR2. 1 by digestion with BamH1 and Not1 and cloned into analogous sites of a pIRES(neo) vector (Clontech, Palo Alto, Calif.) containing a neomycin selection cassette.

Forward and reverse primers were made to a 5′-flanking region of CYP3A4, known to contain the PXRE (Quattrochi et al., J. Biol. Chem. 270:28917-28923 (1995)). The forward and reverse oligonucleotide sequences were 5′-AGACTCACCTCTGTTCAGGGAAA-3′ (SEQ ID NO:3) and 5′-CACCTTGGAAGTTGGC-3′ (SEQ ID NO:4) respectively. This 480 base pair region was amplified by PCR from genomic DNA isolated from a sample of human liver. The amplimer was cloned into pCR2.1 and sequenced. The enhancer region was then liberated from pCR2.1 with EcoR1, blunt-ended and subsequently cloned into the Sma1 site of the pGL3-promoter vector (Promega, Madison, Wis.) without a mammalian selectable marker and including a luciferase reporter gene. Sequence analysis verified that the enhancer was identical to that previously published (Quattrochi et al., J. Biol. Chem. 270:28917-28923 (1995)) and that the oligonucleotide had been inserted.

Stable Transfections and Selections of G4 1 8-Resistant Colonies

HepG2 cells were harvested at approximately 50% confluency and seeded in six well dishes at 5×10⁵ cells per well in DMEM containing 10% fetal bovine serum (FBS). After twenty four hours recovery, cells were transfected with the following combinations: CYP3A4 enhancer/pGL3promoter and hPXR/pIRES(neo) at a ratio of 5:1 (six micrograms total DNA/well), CYP3A4 enhancer /pGL3 promoter and pIRES(neo) (5:1 ratio, six micrograms DNA per well), pGL3promoter and pIRES(neo) (5:1 ratio, six micrograms DNA/well) and pGL3promoter and hPXR/pIRES (5:1 ratio, six micrograms of DNA per well) using a modification of the calcium phosphate co-precipitation procedure (Ausubel et al., Current Protocols in Molecular Biology, Green Publishing Associate/Wiley Interscience, N.Y. (1990)). The control cells were those that received plasmid DNA containing pGL3 promoter and pIRES(neo) or pGL3promoter and HPXR in pIRES(neo). After sixteen hours of exposure to the precipitated DNA, the culture medium was removed, cells washed twice with DMEM, and fresh media containing 10% FBS added. After an additional twenty four hours, media was replaced with that containing 400 micrograms per milliliter of G4 18. Media was changed every two days for three weeks until small colonies were visible. Single colonies were selected and transferred to twenty four well Costar plates (VWR, Westchester, Pa.). Each of the twenty four wells contained the same media and cells were grown to confluency with media changes every three days. Confluent wells were trypsinsed and cells transferred into six well plates and upon reaching confluency therein, cells were further transferred to T75 flasks. Confluent flasks of randomly selected colonies were trypsinzied and used to seed 96 well plates to measure rifampicin-induced luciferase response of individual colonies to test for the presence of recombinants.

Luciferase Assay

Luciferase assays were performed as specified by the manufacture (LucLite system, Packard Instrument, Meriden, Conn.). Activity was determined using the Packard LumnniCount luminometer and results expressed as relative light units or fold increase above control (DMSO treated cells).

Treatment of Stably Transformed Cells

The HepG2 derived cell lines containing recombinant DNA were grown as monolayers in media including Dulbeccos's Modified Eagle's Medium (DMEM, Gibco/BRL, Gaithersburg, Md.), 50 U/ml penicillin, 100 micrograms per milliliter streptomycin, 0.1 milli molar non-essential amino acids (Gibco/BRL), 0.4 milligrams per milliliter G418 (Gibco/BRL) 10% fetal bovine serum (FBS, Hyclone. Logan, Utah) and maintained in an atmosphere of 5% CO₂ and 95% air at 37° C. Cells were seeded in T75 flasks and grown to confluency. After three to five days, cells were removed from flasks by trypsinization and replated on 96 well plates at a density of about 1.0×10⁴ cells per well in DMEM media containing 0.1% FBS and G418 but without indicator (phenol red). After a seventy two hour recovery, the hepatomas, control and CYP3A4 enhancer containing cells and those with hPXR+3A4 enhancer, were treated with 0.1% DMSO (control) or inducer dissolved in DMSO for various time periods and concentrations in fresh media containing 0.1% FBS and G418 without indicator. That cells contained the CYP3A4 enhancer was verified by comparing results to control cells transfected with hPXR/pIRES(neo) and pGL3promoter or pIRES(neo) and pGL3promoter. Screening for cells containing the pGL3/3A4 enhancer were performed by treatment with ten micro molar rifampicin and 0. 1% DMSO. Those cells exhibiting greater than three-fold increases in luciferase activity above control (DMSO-treated) cells were considered transformed with the correct plasmids. Finally, copy number of the 3A4 enhancer integrated into the genome of the HepG2 cells was verified by Southern blot analysis.

To test the cell lines considered positive for the pIRES(neo) and pGL3/3A4 or HPXR+pGL3/3A4, time course studies were performed. Cells were treated with ten micromolar rifampicin for six to seventy eight hours, with analysis of response determined at six hour intervals. In addition, dose response curves were constructed for various CYP3A4 inducers and non-inducers to confirm the specificity of the response element. The dose-response curves consisted of concentrations ranging from 1 to 1,000 micro molar at five different doses. The agents tested were RU486 (Biomol, Plymouth Meeting, Pa.), mevastatin (Biomol), rifampicin (Sigma Chemical, St. Louis, Mo.), omeprazole (Astra-Zeneca, Sweden), clotrimazole (Sigma), phenobarbital (Merck, West Point, Pa.), or dexamethasone (Sigma) and as negative inducers, pregnenolone 16(alpha)-carbonitrile (PCN, Sigma) and TCDD (Chemsyn Science Laboratories, Lenexa, Ky.). Cells were exposed to each compound for 72 hours. All inducers were dissolved in DMSO (Sigma Chemical, St. Louis, Mo.) and this solvent was added to control cells at 0.1%.

B. Results

Identification of G41 8-Resistant Colonies Expressing Inducible Luciferase Activity

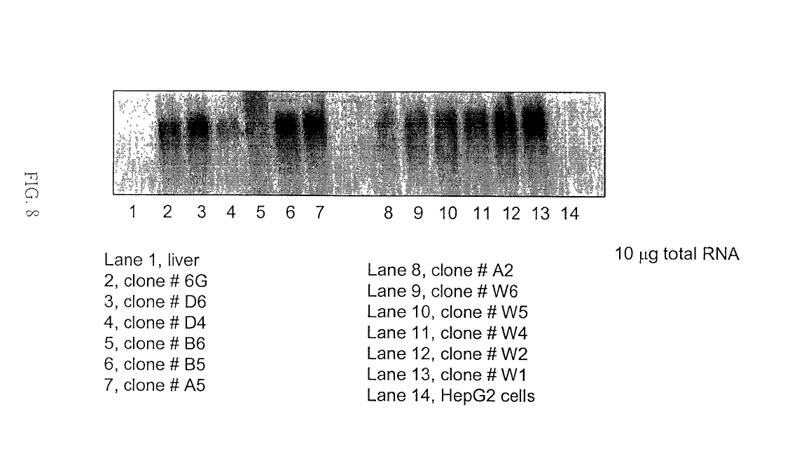

Stable cell lines were developed by transfection of the plasmids, p3A4-enhancer, phPXR, and control vectors into HepG2 cells and selecting from G418 resistance. Resistant colonies were identified for the p3A4-enhancer, p3A4-enhancer-phPXR, and control vectors (TABLE 1). Southern blot analysis of total cellular DNA from several transformants confirmed the presence of stably integrated CYP3A4 enhancer sequences. Validation that hPXR was stably integrated into cells receiving this plasmid was by Northern blot analysis of several colonies (FIG. 8). When compared to RNA from HepG2 cells not transformed with phPXR or primary cultures of human hepatocytes, PXR mRNA was significantly over-expressed. Randomly selected colonies were tested for inducible luciferase activity by treatment with DMSO or ten micro molar rifampicin. TABLE 1 summarizes the number of G418-resistant colonies screened for luciferase activity and the number of G418-resistant colonies having either basal or induced luciferase activity.

TABLE 1

| | Number of colonies | Number of Colonies With: | |
|---|---|---|---|
| | screened for luciferase | Basal luciferase activity | Induced luciferase |
| DNA used in transfection | activity | (a) | activity (b) |
| p3A4 enhancer | 13 | 11 | 6 |
| p3A4 enhancer and | 96 | 79 | 36 |
| phPXR | | | |
| Control | 6 | 3 | 0 |
| (pluciferase plus phPXR) | | | |
| HepG2 cells were harvested at approximately 50% confluency and seeded in 6-well dishes at about 5 × 10⁵ cells per well in DMEM containing 10% FBS. After twenty four hours recovery, cells were transfected. After an additional forty hours, media was replaced with that containing 400 micrograms per milliliter G418. Media was changed every two days for three weeks until small colonies were visible. Individual G418-resistant colonies | | | |
| # were expanded and tested for recombinants by treatment with 10 micro molar rifampicin, followed by analysis of luciferase activity. | | | |

(a) Defined as four times background.
(b) Defined as the ratio of rifampicin-treated to DMSO-treated.

Transfection of the p3A4-enhancer plasmid into HepG2 cells, followed by G418 selection, resulted in the isolation of several G41 8-resistant colonies, of which thirteen were tested for luciferase activity. Eleven G418-resistant colonies were able to support basal-level luciferase expression and six colonies supported inducer-mediated luciferase activity when treated with ten micro molar rifampicin for forty-eight hours or seventy-two hours.

Thirty-six G41 8-resistant colonies containing the stably integrated p3A4-enhancer and phPXR plasmids showed high levels of luciferase expression with rifampicin treatment (TABLE 1). Three control G418-resistant colonies (clones) harboring the pIRES(neo)+pluciferase plasmids exhibited basal level luciferase activity. The p3A4-enhancer+phPXR and p3A4-enhancer transformants containing the highest inducible luciferase activity were chosen for further studies and designated PXR/3A4 (colony IF) and 3A4 (colony 13), respectively.

Inducible Luciferase Activity from Stably Integrated CYP3A4 Sequences

The initial experiments performed in 96 well plates consisted of a time response curve for the p3A4-enhancer+phPXR, p3A4-enhancer, and the vector control cells. Exposure to ten micro molar rifampicin ranged from zero to seventy two hours (FIG. 9). For colony 3A4/13, rifampicin-mediated induction of luciferase activity was apparent at seventy two to seventy eight hours following exposure and ranged from 35 fold to 43 fold above cells treated with DMSO.

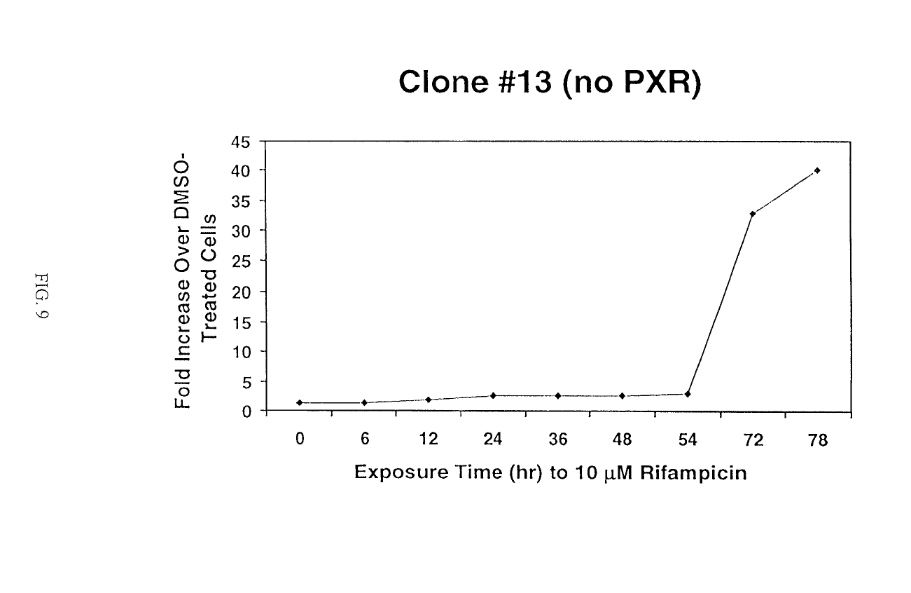

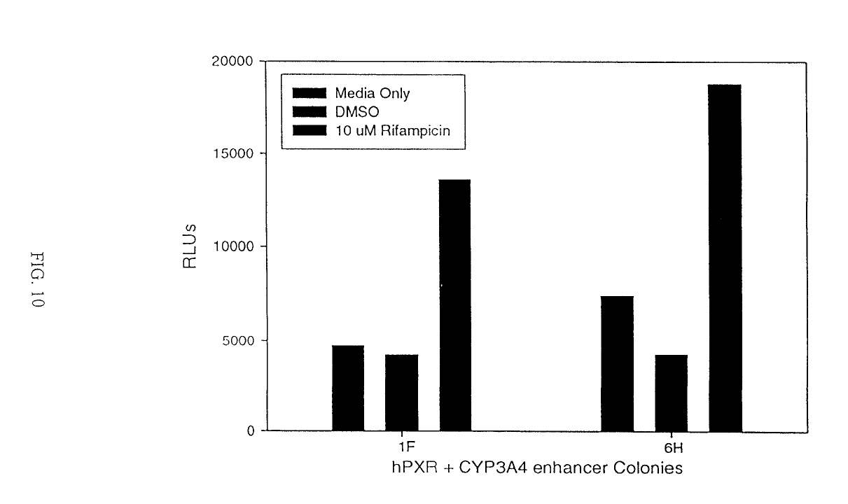

Two separate colonies containing CYP3A4 enhancer and hPXR, colonies IF and 6H, exhibited luciferase activity that was 2.8 to 3.8 fold above DMSO treated cells upon seventy two hours of exposure to ten micro molar rifampicin (FIG. 10). In addition, various amounts of cells were added to each well to determine the preferred or optimal amount, for example gave the greatest response with the least background (FIG. 11). This number (fifty micro liters) reflected the amount of cells needed to produce a readily detectable luciferase signal, low background levels, and that amount that would not alter the pH of the media over a seventy-two hour drug exposure period.

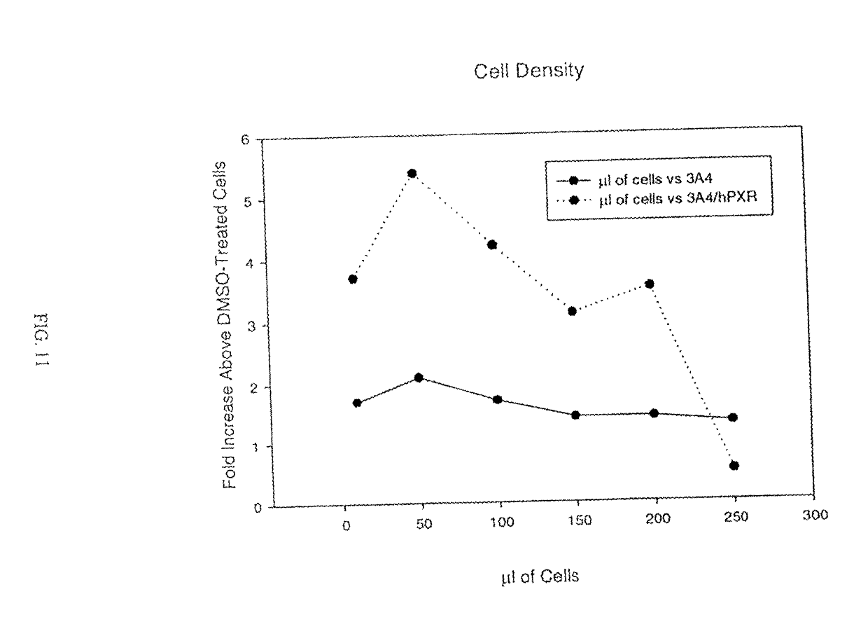

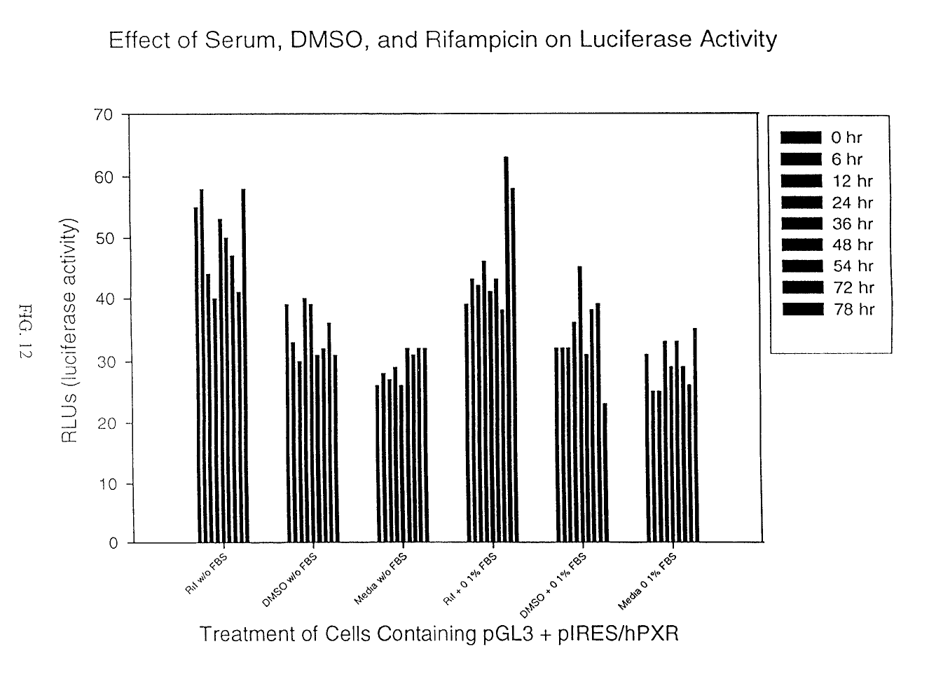

Finally, whether serum had an affect on background luciferase activity was tested using a control transformant containing the phPXR+pGL3promoter (FIG. 12). The results also indicate that serum did not alter luciferase activity.

Induction of CYP3A4 in Human Hepatocytes

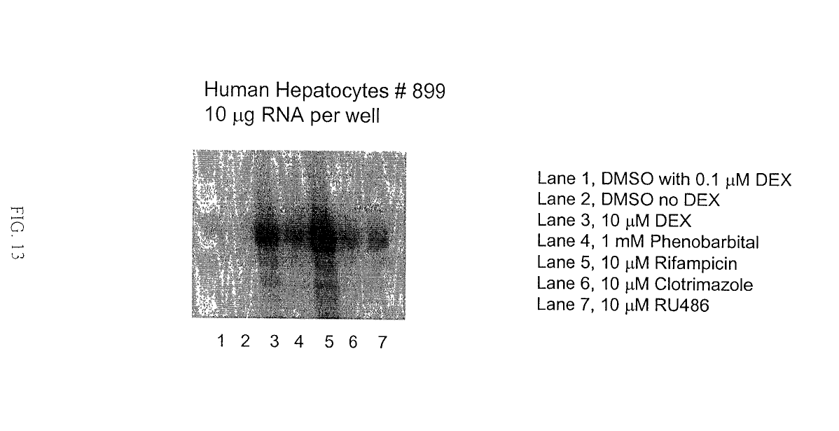

Human hepatocytes were treated with various CYP3A4 inducers for forty eight hours, harvested, and RNA and cell homogenates isolated. FIG. 13 depicts the results of Northern blot analysis on RNA from primary cultures treated with various inducers including dexamethasone (ten micro molar), phenobarbital (one milli molar), rifampicin (ten micro molar), clotrimazole (ten micro molar), and RU486 (ten micro molar). Results indicate that cells exposed to media without dexamethasone did not express CYP3A4. In 0.1 and 10 micro molar dexamethasone, CYP3A4 levels are apparent. Indeed, ten micro molar dexamethasone significantly increased eight-fold CYP3A4 mRNA. Moreover, rifampicin produced a 7.8-fold increase in 3A4 message above that observed in cells exposed to 10⁻⁷ M dexamethasone in 0.1% DMSO. Whereas phenobarbital, clotrimazole and RU486 slightly increased CYP3A4 message 3.8-fold, 4.9-fold and 1.7-fold, respectively, over 0.1% DMSO and 10⁻⁷ M dexamethasone treated cells.

High Throughput System Containing Stable Cell Lines

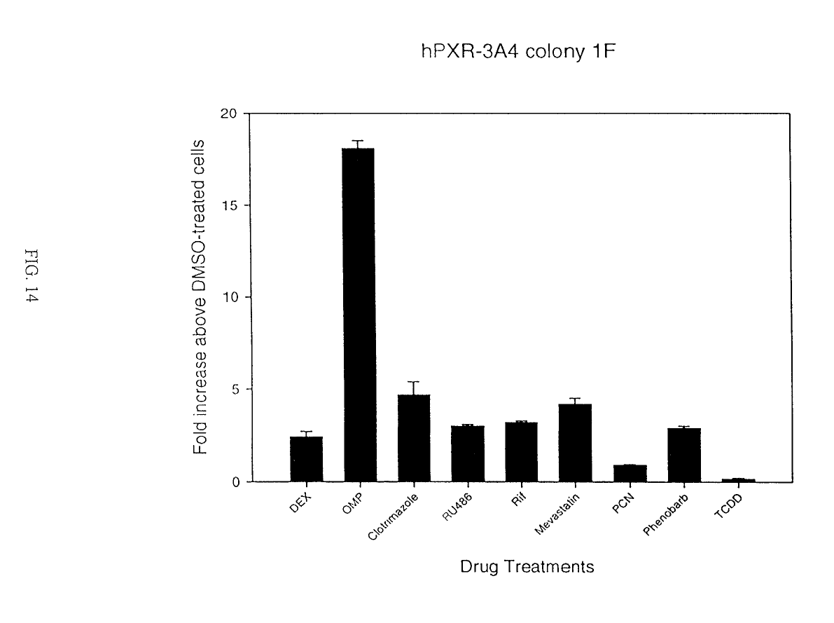

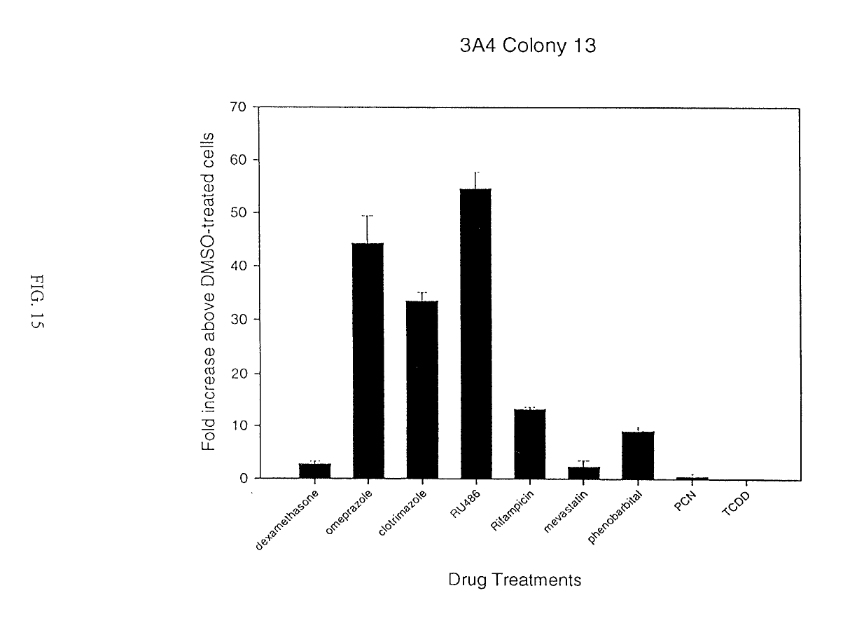

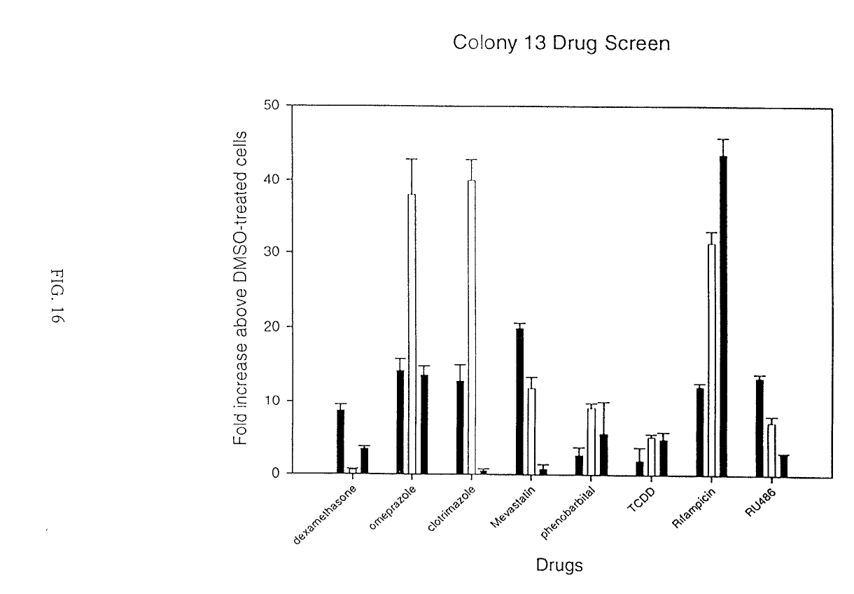

Using the 96-well plate high throughput format, various inducers and non-inducers of CYP3A4 were examined. Each chemical was applied to the cells at different concentration in quadruplicate. Both cells containing the PXR+3A4 enhancer and those with only the 3A4 enhancer (without exogenous hPXR, colony 13)were examined. FIG. 14 and FIG. 15 depict the change in luciferase activity in stably transformed cells (colony IF) harboring both p3A4 and phPXR treated with various known CYP3A4 inducers and two non-inducers, namely TCDD and PCN at single concentrations. At single concentrations, omeprazole appeared to produce the largest response when compared to the other inducers, while PCN and TCDD produced minimal luciferase activity, less than two-fold. Colony 13 harboring the 3A4 enhancer and luciferase produced greater fold increases in luciferase activity for all inducers when compared to colony 1F. Omeprazole, clotrimazole and RU486 produced the largest induction while PCN and TCDD produced less than one-fold increase. When three different concentrations of each inducer were tested in colony 13, 100 micromolar omeprazole produced the largest induction. Rifampicin (25 micromolar) plus 10 micromolar clotrimazole also produced between 40-fold and 45-fold increase (FIG. 16). These results indicate that cell lines harboring the CYP3A4 enhancer are efficient at screening inducers and that the addition of hPXR in constructing the stable transformants does not increase the induction of CYP3A4.

### Example II

In this example, the effects of several agents, such as dietary flavonoids, on CYP1A1 expression utilizing a high throughput screening system for assessing human CYP induction, are examined. HepG2 cells stably integrated with regulatory regions of human CYP1A1 were treated with resveratrol, apigenin, curcumin, kaempferol, green tea extract (GTE), (−) epigallocatechin gallate (EGCG), quercetin, and naringenin. Of these flavonoids, resveratrol produced the largest increase in CYP1A1-mediated luciferase activity (ten-fold) whole GTE, apigenin, curcumin and kaempferol produced two-fold to three-fold increases in activity. In caparison to TCDD, omeprazole or benzanthracene, where increases in luciferase activity ranged from twelve to thirty-five fold, these flavonoids exhibited weak agonist activity. The remaining compounds, EGCG, quercetin, and naringenin produced negligible effects. Cotreatment of cells with TCDD and GTE, naringenin and apigenin resulted in fifty-eight, seventy-seven and seventy-four percent reductions, respectively, in TCDD-mediated CYP1A1 induction, indicating that these flavonoids exhibit potential antagonist activity towards the Ah receptor. Furthermore, results indicate that GTE and apigenin possess Ah receptor antagonist and weak agonist activities. Also disclosed is a 96-well plate assay for high throughput screening for P450 induction in less than twenty-four hours, was efficient for determining the effects of flavonoids on human CYP1A expression. Signal to noise ratios were low and well-to-well and replicate variability was below ten percent allowing induction to be easily detected in this system. These features illustrate the reliability and feasibility of this high volume screening system for identifying CYP inducers. Furthermore, results produced with the stable cell line were corroborated in HepG2 cells and primary cultures of human hepatocytes, indicating that stably integrated cell lines harboring enhancer elements of a P450 gene can be utilized in high throughput screening systems.

A. MATERIALS AND METHODS

Cell Cultures and Treatment

Cell line 101L (University of California San Diego), derived from human hepatoma cell line HepG2 (ATCC, Wistar Institute), was stably transfected with the human CYP1A1 promoter and the 5′ flanking sequences linked to the luciferase reporter gene (see, Postlind et al., Toxicol.

Appl. Pharmacol. 118:255-262 (1993)). Briefly, the 101L cell line was established by stably transfecting a plasmid containing the human CYP1A1 promoter (−3275 to +89) linked to the firefly luciferase reporter gene into the human hepatoma cell line, HepG2. The CYP1A1 promoter region contains three DREs ad the cell line was estimated to contain two copies of the integrated plasmid.

The 101L cell line was grown as monolayers in media including Dulbecco's Modified Eagle's Medium (DMDM, Gibco/BRL), 50 U/ml penicillin, 100 micrograms/ml streptomycin, 0.1 millmolar essential amino acids (Gibco/BRL), 0.4 milligrams/ml G418 (Gibco/BRL), 10% fetal bovine serum (FBS, Hyclone, Logan Utah) and maintained in an atmosphere of 5% CO₂ and 95% air at 37° C. Cells were initially seeded in flasks containing media without G418. After an overnight incubation the cultures were changed into media containing G418 for antibiotic selection. After three to five days, cells were removed from flasks by trypsinization and replated on either a twenty-four-well plate at a density of 3.5×10⁵ cells per well, or a ninety-six-well plate at a density of 7.5×10⁴ cells per well, in DMEM media that was replaced with that containing 0.1% FBS and without G418 or indicator (phenol red). The next day, media containing 0.1% FBS and G418 was added to the cultures. After twenty-four hours, cells containing stably integrated reporter constructs were treated with 0.1% DMSO (control), 2,3,7,8-tetrachlorodibenzo-p-dioxin (TCDD, Chemsyn Science Laboratories, Lenexa, Ky.), 3-methylcholanthrene (3-MC, Sigma Chemical Co., St. Louis, Mo.), benzanthracene (BA, Sigma), omeprazole (Astra-Zeneca, Sweden), rifampicin (Rif, Sigma), quercetin (Sigma), green tea extract (GTE, Sigma), resveratrol (Sigma), apiginen (Sigma), curcumin (Sigma), kaempferol (Sigma), (−)-epigallocatechin gallate (EGCG, Sigma), or naringenin (Sigma) in fresh media containing 0.1% FBS and G418 without indicator. For the antagonist experiments, cells were co-treated with a flavonoid and two nanomolar TCDD. All inducers were dissolved in DMSO and this reagent was added to control cells at 0.1%. The cells were treated with varous doses and times (six to eighteen hours). After treatment, the media containing the compound was removed by aspiration and replaced with one-hundred microliters per well of DMEM for direct analysis of luciferase activity. Experiments were performed on 101L cells from frozen stocks on the initial derivation and the passage number was limited to thirty. The latter passages exhibited responses similar to those of the earliest passage.

Luciferase Assay

Luciferase assays were performed as specified by the manufacturer (LucLite system, Packard Instrunent, Meriden, Conn.). Activity was determined using a Packard LumiCount luminometer and results expressed as relative light units or fold increase above control (DMSO treated cells.

HepG2 Cultures and Treatment

HepG2 cells were obtained from American Type Culture Collection (ATCC). Cells were grown in DMEM (Gibco/BRL). Twenty-four hours after cells were plated and grown to confluency, they were treated with one of the bioflavonoids, TCDD, or beta-naphthoflavone (Sigma). All inducers were dissolved in DMSO and this solvent was added to control cells at 0.1%.

Primary Cultures of Human Hepatocyte and Treatment

Six well plates containing human hepatocytes were obtained from the Liver Tissue Procurement and Distribution System (LTPADS, University of Minnesota, Minneapolis, Minn.). Upon arrival, media was replaced with that containing Human Hepatocyte Maintenance Media (HHMM, Clonetics, San Diego, Calif.) (Runge et al., Biochem. Biophys. Res. Commun. 273:333-341 (2000)) and maintained in an atmosphere of 95% air and 5% CO₂ at 37° C. The following 20 day, cells were treated for twenty-four hours with 0.1% DMSO (control), 50 micromolar benzanthracence, 2 nanomolar TCDD, 20 micromolar kaempferol, 20 micromolar resveratrol, or micromolar naringenin, 10 micromolar apigenin, 0.1 milligrams/ml GTE or co-treated with TCDD and a flavonoid. All inducers were dissolved in DMSO and added to media at a 0.1% final concentration of this reagent. After treatment, media was removed and cells harvested for RNA isolation.

RNA Isolation and Northern Blot Analysis

Total RNA from hepatocytes or HepG2 cells was isolated using Trizol™ reagent (Gibco BRL Products, Gaithersburg, Md.) and quantified by measuring absorbance at 260 nm; purity was assessed by determining the 260/280 nm ratio. Northern blot analysis was performed by electrophoresis of total RNA (10 micrograms) through a 1% agarose-2.2 M formaldehyde gel, followed by blotting onto a nylon membrane (MSI, Westboro, Mass.) (Shih et al., Hum. Exper. Toxicol. 18:95-105 (1999)). RNA was cross-linked to the membranes using a UV Crosslinker (Stratagene, La Jolla, Calif.) and the membranes hybridized to random-primed cDNA probes encoding human CYP1A1. The cDNA probe for human CYP1A1 has previously been described (Shih et al., Hum. Exper. Toxicol. 18:95-105 (1999)). A cDNA probe for human 18S RNA probe (Ambion, Austin Tex.) was used to normalize the amount of RNA loaded in each lane. Hybridization of blots was performed as previously described (Quattrochi et al., DNA 4:395-400 (1985)). Autoradiographs of Northern blots were quantified by densitometry using a Model GS-670 Imaging Densitometer equipped with Molecular Analysis/Mac version 1.1.1. Image Analysis software (BioRad laboratories, Hercules, Calif.) or by scaning autoradiograms with a ScanMaker II (Microtek) and digitized with Un-Scan-It software (Silk Scientific, Orem Utah). Exposure times used were in the linear range of the film, Kodak XAR-5.

Data Analysis

Student's t test was used for the statistical analysis of data. Statistical significance was defined at a level of p<0.05. Data are expressed as the mean +/− standard deviation (SD).

B. Results

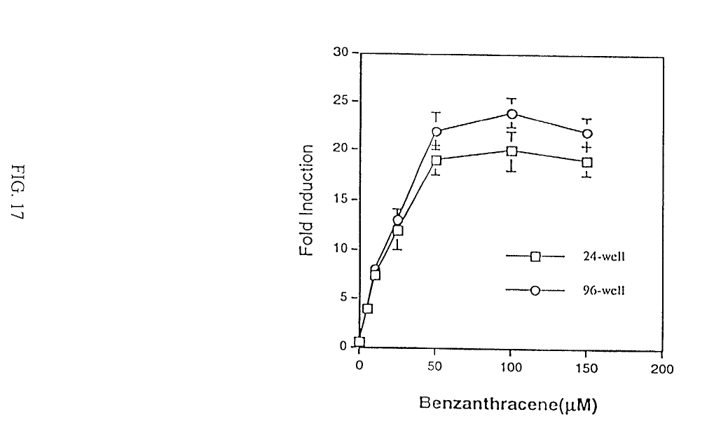

101L cells were plated at a density of 3.5×10⁵ or 7.5×10⁴ cells per well in twenty-four or ninety-six-well plates, respectively. Following exposure to the Ah receptor ligand, benzanthracene, luciferase activity was determined. When results obtained from ninety-six well plate assays were compared to those from twenty-four plates, negligible differences in luciferase activity were detected (FIG. 17). These findings alleviated the concern that too few cells per well would produce an inadequate signal. There was also concern that variability between replicate wells would be high. However, the ninety-six well format exhibited a maximum of 10% well-to-well variability with minimum background (FIG. 17). The following experiments were performed using the ninety-six well format.

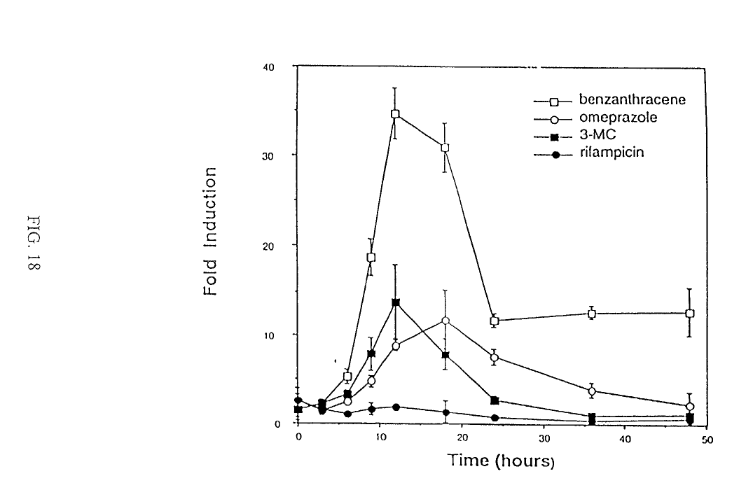

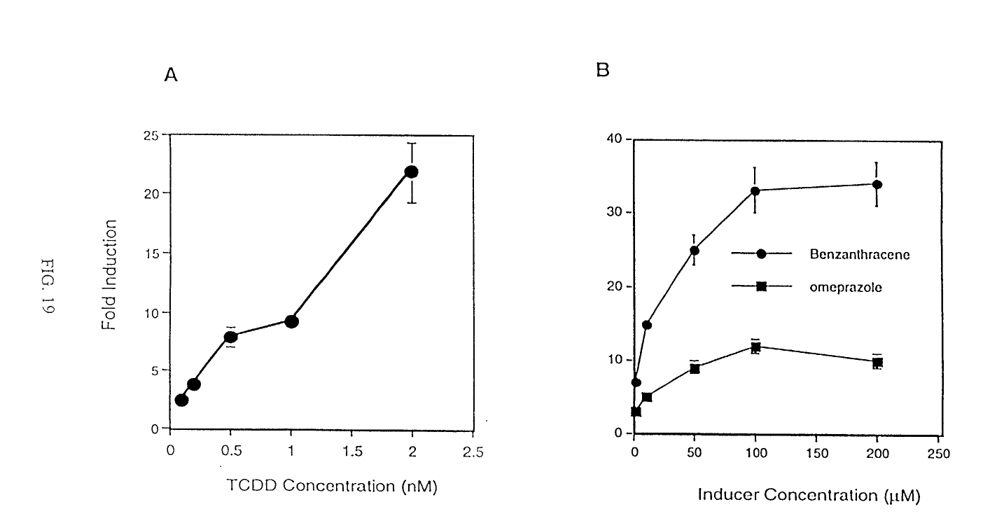

The maximal time period for inducer exposure was determined by establishing a time course of inducer-mediated luciferase activity. Enhanced activity was observed within six hours of dosing with benzanthracene (100 micromolar), omeprazole (100 micromolar) or 3-MC (10 micromolar (FIG. 18). Maximum induction by benzanthracene (thirty-five fold) and 3-MC (fourteen fold) occurred at twelve hours while omeprazole mediated induction was maximal at eighteen hours (twelve fold), after which luciferase activity declined. The decline in inductive response was more than likely due to metabolism of the inducer by HepG2 CYP1A1. As expected, induction by rifampicin (100 micromolar) was negligible because this antibiotic is not known to be a CYP1A inducer (Kostrubsky et al., Drug. Metab. Dispos. 27:887-894 (1999)). These results indicate that this high-volume screening procedure is effective at monitoring easily detectable induction within a relatively short time period, for example less than twenty-four hours. In addition, the concentration dependent effects of various known CYP1A1 inducers were determined in this system. Dose response curves ranging from 0.5 to 2.5 nanomolar were generated for TCDD (FIG. 19A), and 1 to 200 micromolar for benzanthracene and omeprazole (FIG. 19B). For benzanthracene and omeprazole, maximum induction (thirty-five fold and twelve fold, respectively) occurred at 100 micromolar. The fold induction by TCDD had not peaked at a dose of 2 namomolar, and this dose produced a twenty-two fold increase in luciferase activity.

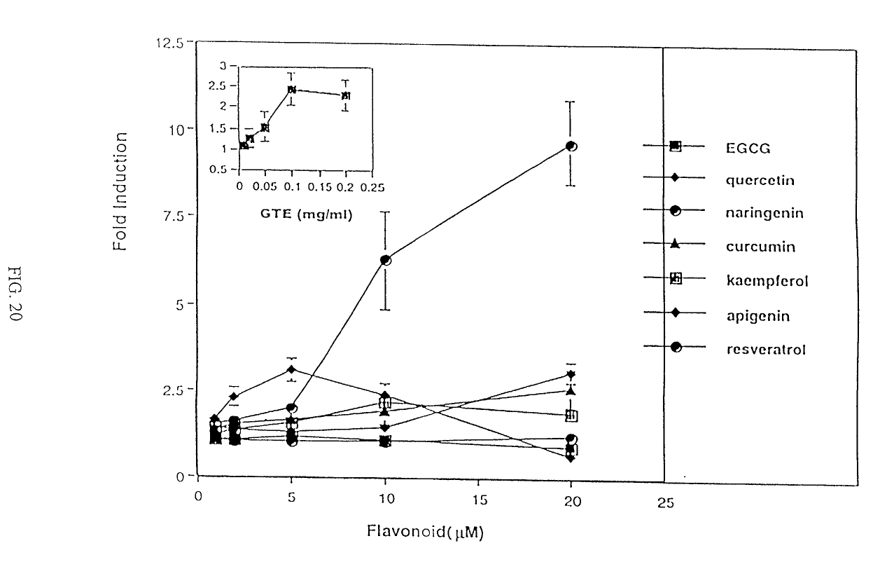

The use of high throughput methods for mechanistic studies were also investigated. To determine a mechanism that may be involved in flavonoid prevention of chemical carcinogenesis, we examined the effects of several dietary flavonoids on CYP1A1 induction. Results of these studies could indicate if the flavonoid exhibitied Ah receptor agonist and/or antagonist activities. Initial studies examined the ability of various naturally occurring flavonoids to induce CYP1A1-promoter-mediated reporter gene activity in the 101L cell line. Dose response curves for GTE, EGCG, quercetin, curcumin, kaempferol, naringenin, apigenin and resveratrol were determined. Of these flavonoids, resveratrol (10 micromolar) produced the largest induction of CYP1A1 (ten-fold). The second most effective flavonoid inducers were apigenin, uercetin and curcumin (three fold). A three-fold elevation in luciferase activity was observed with five micromolar of apigenin treatment, whereas higher doses of quercetin and curcumin (twenty micromolar) provided for similar levels of induction. Doses higher than five micromolar apigenin produced a decline in CYP1A1 induction, which more than likely is the result of cytotoxicity. GTE (0.1 milligrams/ml) (FIG. 20, inset) and kaempferol also produced slight induction (2 to 2.5 fold induction) on CYP1A1-promoter-mediated induction of luciferase activity at concentrations ranging from 1 to 20 micromolar (FIG. 20).

To validate similar inductive responses of the endogenous CYP1A1 gene, HepG2 cells were also treated with the same flavonoids. Enhanced CYP1A1 mRNA expression was observed in cells treated with GTE (10% of TCDD induction (TABLE 2). Although increased expression of CYPA1 mRNA occurred with these flavonoids, the induction was much less than that of beta-naphthoflavone (50% of TCDD response). Collectively, GTE, resveratrol and apigenin appear to be weak agonists for the Ah receptor.

TABLE 2
The Effect of Flavonoids on CYP1A1 mRNA Leves in HepG2 Cells

| | HepG2 CYP1A1 mRNA (%) |
|---|---|
| Treatment | (a) |
| TCDD | 100 |
| 50 micromolar beta-napthoflavone | 50 |
| 100 micromolar beta-napthoflavone | 53 |
| Resveratrol | 12 |
| GTE | 10 |
| Apigenin | 1 |
| Naringenin | 0 |
| TCDD + Resveratrol | 86 |
| TCDD + Apigenin | 43 |
| TCDD + Naringenin | 30 |

(a) CYP1A1 mRNA levels were normalized to TCDD induction (100% increase in CYP1A1 mRNA). Each value represents the mean of two separate determinations that differed by <10%.

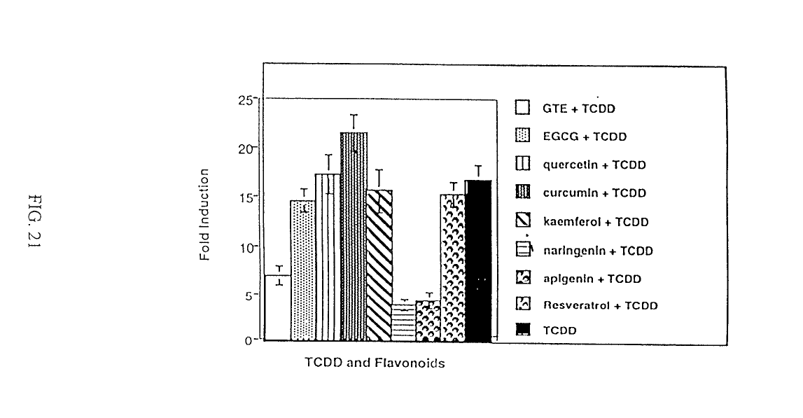

The ability of flavonoids to exhibit Ah receptor antagonism activity was talso examined using this high throughput screening system. Co-treatment of the 101L cells with TCDD and flavonoids in the ninety-six well plate assay resulted in decreased TCDD-mediated induction of reporter gene activity by some of the flavonoids, indicating that certain of these dietary agents exhibited antagonist activity (FIG. 21). When the 101L cells were co-treated with GTE and TCDD a 58% reduction in luciferase activity was observed compared to cells treated with TCDD alone. Furthermore, the flavonoids naringenin and apigenin produced a 77% and 74% reduction, respectively, in TCDD mediated induction. Results of these studies demonstrate that these dietary flavonoids are capable of antagonizing TCDD-mediated induction of CYP1A1 promoter activity, with naringenin having the greatest effect (FIG. 21). Based on results where apigenin or GTE alone displayed a 2.5 to 3-fold induction of CYP1A1-mediated reporter gene activity (FIG. 20), these flavonoids appear to exhibit agonist and antagonist activity toward the Ah receptor.

The other flavonoids either produced no appreciable change in TCDD-mediated induction of luciferase activity or stimulated its effects. Indeed, co-treatment with TCDD and curcumin produced a 1.5-fold stimulation above the effects of TCDD alone, indicating that mechanisms in addition to those involving the AhR may play a role in induction of the P450 by curcumin. When HepG2 cells were co-treated with TCDD and individual flavonoids, results similar to those obtained with the reporter gene assay were observed. Resveratrol produced a slight decrease in the TCDD inductive response of CYP1A1 mRNA (14% reduction) whereas apigenin and naringenin produced significant reductions in CYP1A1 mRNA accumulation mediated by TCDD (57% to 70% decreases (TABLE 2). These results corroborate those produced in the 101L cell line and suggest that apigenin and naringenin have AhR antagonist activity.

To demonstrate if similar effects would occur in primary cultures of human hepatocytes, Northern analyses were performed on mRNA isolated from these cells trreated with TCDD, flavonoids, or a combination of TCDD and individual flavonoids. Results revealed similar findings to those produced by the high throughput or high volume screening system. Resveratrol enhanced CYP1A1 mRNA levels to 5% and 12% of TCDD induction in hepatocytes from two individual liver samples (Subject A and Subject C) while GTE enhanced CYP1A1 mRNA levels to 34% of TCDD induction in one culture (Subject A) (TABLE 3). In comparison, 100 micromolar benzanthracene caused induction of CYP1A1 mRNA to 50% of that observed with TCDD in all subjects. In hepatocytes from one subject, not only benzanthracene, but also resveratrol, apigenin and kaempferol produced accumulation of CYP1A1 mRNA (Subejct C, TABLE 3). Resveratrol increased expression to 12%, apigenin to 3% and kaempferol to 10% of that observed with benzanthracene. Hepatocytes from two other subjects (Subject B and Subject D, TABLE 3) did not display CYP1A1 induction with any of the flavonoids, but did exhibit CYP1A1 mRNA accumulation produced by TCDD and benzanthracene (50% of TCDD levels).

Human hepatocytes were also co-treated with TCDD and individual flavonoids. Resveratrol produced a 49% reduction in enhanced levels of CYP1A1 mRNA produced by TCDD. Apigenin and naringenin produced 78% and 80% reductions, respectively, in TCDD-mediated increases of CYP1A1 mRNA (TABLE 3). These results were similar to those obtained from co-treatment of the 101L cell line with TCDD and apigenin or naringenin (FIG. 21).

TABLE 3
The Effect of Flavonoids on CYP1A1 mRNA Levels in
Primary Cultures of Human Hepatocytes

| | Subject A (%) | Subject B (%) | Subject C (%) | Subject D (%) |
|---|---|---|---|---|
| Treatment | (a) | (a) | (b) | (a) |
| TCDD | 100 | 100 | ND | 100 |
| Benzanthracene | 50 | 50 | 100 | 52 |
| GTE | 34 | 0 | ND | 0 |
| Resveratrol | 5 | 0 | 12 | 0 |
| Apigenin | ND (c) | 0 | 3 | 0 |
| Kaempferol | ND | 0 | 10 | 0 |
| Naringenin | ND | ND | ND | 0 |
| TCDD + | ND | ND | ND | 51 |
| Resveratrol | | | | |
| TCDD + Apigenin | ND | ND | ND | 22 |
| TCDD + | ND | ND | ND | 20 |
| Naringenin | | | | |

(a) CYP1A1 mRNA levels were normalized to TCDD induction (100% increase in CYP1A1 mRNA)
(b) CYP1A1 mRNA levels were normalized to benzanthracene induction (100% increase in CYP1A1 mRNA
(c) ND indicates not determined

C. Discussion

This example utilizes a reporter gene assay and a stable cell line, namely 101L cells (Postlind et al., Toxicol. Apl. Pharnacol. 118:255-262 (1993)), to screen potential CYP1A inducers. Stable cell lines harboring P450 enhancers and reporter genes are advantageous for screening applications because the need to continually transfect is alleviated, eliminating variability associated with transient transfections. Stably integratred cells also markedly increase sensitivity allowing induction to be easily assessed. Consistent results are obtained and the stable cells allow an alternative to other systems that are time consuming and labor intensive. Thus, the use of stable cell lines with P450 enhancers can facilitate screening of potential inducers. Indeed, the 101L reporter gene system is an application currently being used in 6 well plate formats by industry to screen environmental samples for the presence of CYP1A1-inducing compounds (Jones et al., Environ. Toxicol. Pharmacol. 8:119-126 (2000)).

To develop a high throughput system with stable cell lines, the previously characterized 101L cells were initially plated in either 24 well or 96 well plates having a standard footprint and treated with benzanthracene (FIG. 17). Results generated from these experiments indicated that the 96 well plate format was as efficient as the 24 well plate format. Furthermore, in the high throughput (96-well) format, there was minimum background and less than 10% well-to-well variability. In the presence of various CYP1A inducers, maximum induction (12 to 35-fold) occurred within a 24 hour exposure period, similar to that obtained in 6-well plates (Postlind et al., Toxicol. Appl. Pharmacol. 118:255-262 (1993); Quattrochi and Tukey, Mol. Pharmacol 43:504-508 (1993)). Ziccardi et al. (Toxicol. Sci. 54:183-193 (2000)) reported a 96 well format to screen serum samples for Ah receptor ligands.

To test the high throughput format of the present invention, additional CYP 1A inducers were examined. A dose response curve was established for benzanthracene. Maximum induction in 101L cells previosuly reported in 6 well plates occurred at a dose of 50 micromolar benzanthracene (Jones et al., Environ. Toxicol. Pharmacol. 8:119-126 (2000)). The same dose produced maximum CYP1A1 mediated luciferase activity (33-fold) in the study described herein with the 96-well plate format (FIG. 19B). Other known CYP1A inducers including 3-methylcholanthrene, TCDD and omeprazole also produced induction of luciferase in the 96-well format whereas rifampicin, a CYP3A4 inducer, had no effect (FIG. 17), confuming the specificity of this system to respond solely to CYP1A inducers. TCDD and/or benzanthracene also induced CYP1A1 mRNA in HepG2 cells (TABLE 2) and in all human hepatocyte samples tested. Although not tested here, omeprazole has been shown in previous investigations to induce CYP1A's in human hepatocytes (Dias et al., Gastroenterology 99:737-747 (1990) and Shih et al., Hum. Exper. Toxicol. 18:95-105 (1999)). Collectively, when an inducer produces greater than 12 fold increases in luciferase activity in the high throughput system (HTS), in all likelihood induction of CYP1A1 by the same agent would occur in human hepatocytes.

To determine if this HTS could be used to identify novel CYP1A1 inducing agents, we examined the ability of a variety of dietary flavonoids to induce CYP1A1. Of the flavonoids examined, only resveratrol produced a substantial increase (10-fold) in CYP1A1 mediated-luciferase activity. However, cells treated with concentrations less than 20 micromolar resveratrol had negligible effects on luciferase activity, consistent with previous reports that this agent does not induce CYP1A1 mRNA in breast cancer cell lines or HepG2 cells (Ciolino et al., Cancer Res. 58:5707-5712 (1998) and Casper, Mol. Pharmacol. 56:784-790 (1999)). When induction observed with the reporter gene assay was compared to CYP1A1 mRNA accumulation in primary hepatocytes and HepG2 cells, again resveratrol produced increases in CYP1A1 mRNA from HepG2 cells and in hepatocytes from two individuals (TABLE 2, TABLE 3, particularly Subject A and Subject C). These results indicate that agents producing 10-fold increases in luciferase activity observed in the HTS, could also produce CYP1A1 induction in hepatocytes. Those flavonoids producing 2.5-fold induction or greater in the HTS system, namely GTE and apigenin, also produced slight increases in the accumulation of CYP1A1 mRNA in primary hepatocytes isolated from one of three individuals examined here. Similarly, kaempferol which produces two-fold increases in luciferase activity also caused accumulation of CYP1A1 mRNA in hepatocytes from a single individual. In contrast, quercetin and curcumin did not elicit induction of CYP1A1 mRNA in isolated hepatocytes (data not shown), but did produce moderate increases (2.5 to 3-fold) in luciferase activity. Thus, this disparity in results between the HTS and human hepatocytes among various agents, suggests that when reporter assays exhibit relatively low levels of induction by a particular agent (for example, 2 to 3 fold), increases in primary hepatocyte CYP1A1 may or may not occur.

Based on results obtained here with the HTS, less than 2-fold induction of luciferase activity indicates that increased expression of CYP1A1 would be unlikely to occur in primary hepatocytes. The importance of the hepatocyte finding corroborating those of the HTS lies in the ability to extrapolate human hepatocyte data to the in vivo situation (Ito et al., Annu. Rev. Pharmacol. Toxicol. 38:461-499 (1998) and Kedderis, Chem. Biol. Interact. 107:109-121 (1997)). For example, omeprazole produced induction of CYP1A's in both isolated human hepatocytes (Shih et al., Hum. Exper. Toxicol. 18:95-105 (1999) and Diaz et al., Gastroenterology 99:737-747 (1990)) and in vivo (Rost et al., Clin. Pharmacol. Ther. 52:170-180 (1992)). In general, the pharmacokinetics of xenobiotics have been well predicted from studies with isolated hepatocytes (Kedderis, Chem. Biol. Interact. 107:109-121 (1997)). In this example, good agreement occurred between results generated in the stably transfected cells and human liver cells (primary hepatocytes and HepG2 cells), suggesting that cell lines stably transfected with CYP enchancers would be able to predict the in vivo outcome.

The HTS format for assessing CYP1A1 induction is useful in identifying agents that can elevate expression of CYP1A1 by way of the Ah receptor. Furthermore, this system can be used to determine mechansims involved in CYP induction. This example demonstrates that certain flavonoids were identified as exhibiting weak agonist and/or antagonist activity towards the Ah receptor. With regards to the reliability of this HTS for identifying CYP inducers, signal to noise ratios were low and well-to-well and replicate variability were below 10% allowing induction to be readily detected in this system. Also, results generated with this HTS reflected inducer responses obtained in isolated human hepatocytes or HepG2 cells.

All publications, including patent documents and scientific articles, referred to in this application, including any bibliography, are incorporated by reference in their entirety for all purposes to the same extent as if each individual publication were individually incorporated by reference.

All headings are for the convenience of the reader and should not be used to limit the meaning of the text that follows the heading, unless so specified.

What is claimed is:

1. A cell, comprising:
a first nucleic acid molecule comprising:
a promoter or enhancer operable for a nucleic acid molecule encoding a protein involved in drug metabolism;
a reporter gene,
wherein said promoter or enhancer is operably linked to said reporter gene; and
a second nucleic acid encoding an intracellular receptor or transcription factor, wherein when said intracellular receptor or transcription factor is bound with, associated with or activated by a compound, said intracellular receptor or transcription factor can operably bind with, associate with or activate said promoter or enhancer resulting in the expression of said reporter gene;
wherein when said cell is contacted with a compound that induces the expression of said protein involved in drug metabolism, said reporter gene is expressed.

2. The cell of claim 1, wherein said enzyme involved in drug metabolism is selected from the group consisting of P450s, glucuronosyl transferases, N-acetyltransferases, p-glyoproteins, glutathione transferases and sulfo transferases.

3. The cell of claim 1, wherein said reporter gene encodes an enzyme or a detectable protein.

4. The cell of claim 1, wherein said first nucleic acid molecule is present in an extrachromosomal element.

5. The cell of claim 1, wherein said first nucleic acid molecule is within the chromosome of said cell.

6. The cell of claim 1, wherein said reporter gene is inserted into the chromosome of said cell.

7. The cell of claim 1, wherein said enhancer or promoter is endogenous to the chromosome of said cell.

8. The cell of claim 1, wherein said reporter gene is endogenous to the chromosome of said cell.

9. The cell of claim 1, wherein said intracellular receptor or transcription factor forms a complex with a drug, chemical or metabolite thereof and directly or indirectly produces transcriptional activation of a gene encoding a protein involved in drug metabolism.

10. The cell of claim 1, wherein said intracellular receptor or transcription factor is an orphan receptor or a hormone receptor.

11. The cell of claim 1, wherein said second nucleic acid molecule is present in an extrachromosomal element.

12. The cell of claim 1, wherein said second nucleic acid molecule is present within the chromosome of said cell.

13. The cell of claim 1, wherein said second nucleic acid molecule is endogenous to the chromosome of said cell.

14. The cell of claim 1, wherein said cell is a mammalian cell.

15. The cell of claim 1, wherein said cell is a transformed cell.

16. The cell of claim 1, wherein said cell is a human cell.

17. The cell of claim 1, wherein said cell is a cell line.

18. The cell of claim 1, wherein said cell is from a tissue selected from the group consisting of liver, lung or kidney.

19. A method for evaluating compounds for the property of inducing the expression of a gene encoding a protein involved in drug metabolism, comprising;
providing a test compound;
contacting said test compound with the cell of claim 1; and
detecting the expression of said reporter gene;
wherein expression of said reporter gene is indicative that said compound altered the expression of a gene encoding a protein involved in drug metabolism.

20. The method of claim 19, wherein said method is a high throughput method.
